(19) United States
(12) Patent Application Publication
(10) Pub. No.: US 2011/0105626 A1
(43) Pub. Date: May 5, 2011

(54) USE OF CYCLOHEXANEHEXOL DERIVATIVES FOR THE TREATMENT OF POLYGLUTAMINE DISEASES

(76) Inventor: JoAnne McLaurin, East York (CA)

(21) Appl. No.: 12/594,943

(22) PCT Filed: Apr. 11, 2008
(86) PCT No.: PCT/CA2008/000684

§ 371 Date: Dec. 14, 2010

Related U.S. Application Data
(60) Provisional application No. 60/923,002, filed on Apr. 12, 2007.
(60) Provisional application No. 60/923,120, filed on Apr. 12, 2007.

(51) Int. Cl. A61K 31/047; C07C 35/16; A61P 25/00

(52) U.S. Cl. 514/729; 568/833

(57) ABSTRACT

The present invention relates to methods to modulate the assembly, folding, accumulation, rate of aggregation, oligomerization or clearance of proteins or fragments comprising PoIyQ, by using compositions comprising cyclohexanehexol derivatives. More specifically, the invention provides a medicament comprising cyclohexanehexol derivatives of formula III or formula IV, more particularly a scyllo-inositol compound, analog or derivative thereof, useful in the treatment of polyglutamine diseases, such as Huntington's disease and related neurodegenerative disorders including dentatorubral pallidoluysian atrophy, spinal and bulbar muscular atrophy and spinocerebellar ataxia type 1, 2, 3, 6, 7 and 17.

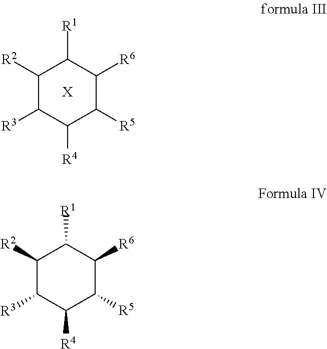

### FIELD OF THE INVENTION

The invention relates to treatment of polyglutamine diseases, and in particular the prevention or inhibition of assembly, disruption, or enhanced clearance of, polyglutamine (PolyQ) aggregates, and/or the improvement of neuron function and/or the prevention of a loss thereof, in individuals in need of such inhibition, disruption, enhancement, improvement, and/or prevention.

### BACKGROUND OF THE INVENTION

A number of neurodegenerative disorders have been found to be caused by expanding CAG triplet repeats that code for polyglutamine. Huntington's disease (HD) is the most common of these disorders. A trinucleotide repeat expansion in the Huntingtin (Htt) gene produces an altered form of the Htt protein with an extended polyglutamine (polyQ) tract [i.e., mutant Huntingtin (mHtt)], that results in intracellular aggregate formation and neurodegeneration. HD inevitably leads to death after a long devastating clinical course characterized by progressive deterioration and irreversible disability. The neuropathological damage characteristic of the disease comprises degeneration of the neurons of the basal ganglia that control involuntary movements (Reiner A., et al, Proc. Natl. Acad. Sci. 85, 5733-37, 1988.). There is no effective treatment for the disease; however, the choreform movements typical of the disease can be reduced, in part, by anti-psychotics or reserpine (Merck Manual, Ed. Merck Res. Laboratories, 17^{th} ed., 1999, 1464).

### SUMMARY OF THE INVENTION

The present invention relates to methods for treating polyglutamine diseases in a subject comprising administering a therapeutically effective amount for treating a polyglutamine disease of a cyclohexanehexyl compound, in particular an isolated and pure cyclohexanehexyl compound, more particularly a scyllo-inositol compound or analog or derivative thereof. The methods of the invention can be used therapeutically or can be used prophylactically in a subject susceptible to polyglutamine diseases.

The invention also provides a method for treating a polyglutamine disease in a subject comprising administering to the subject a therapeutically effective amount of one or more cyclohexanehexyl compound, or a pharmaceutically acceptable salt thereof, or a medicament comprising a cyclohexanehexyl compound and a pharmaceutically acceptable carrier, excipient, or vehicle, which results in beneficial effects following treatment. In particular, the invention relates to a method for the treatment of a subject suffering from a polyglutamine disease comprising administering at least one cyclohexanehexyl compound or a pharmaceutical salt thereof, to the subject in an amount effective to treat the subject.

In an aspect, the invention relates to a method of treatment comprising administering a therapeutically effective amount of one or more cyclohexanehexyl compound, a pharmaceutically acceptable salt thereof, or a medicament comprising a cyclohexanehexyl compound, and a pharmaceutically acceptable carrier, excipient, or vehicle, which upon administration to a subject with symptoms of a polyglutamine disease produces sustained beneficial effects.

In particular aspects, beneficial effects are evidenced by one or more of the following: modulation (e.g., inhibition, reversal, or reduction) of assembly, folding, accumulation, oligomerization, rate of aggregation, oligomerization and/or clearance of proteins or fragments comprising PolyQ, in particular prevention, reduction or inhibition of oligomerization, aggregation and/or assembly of proteins or fragments comprising PolyQ in neurons; reversal or reduction of PolyQ aggregates in neurons after the onset of symptoms of a polyglutamine disease; dissolution and/or disruption of PolyQ aggregates in neurons, and/or enhanced clearance of PolyQ aggregates in neurons; improved neuron function; slowing of degeneration and death of neurons in the brain; increased longevity of a subject; and, slowing or arrest of the progress of a polyglutamine disease.

In an aspect, the invention provides a method of reversing or reducing degeneration of nerve cells in a subject suffering from a polyglutamine disease comprising administering a therapeutically effective amount for reversing or reducing degeneration of nerve cells of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention provides a method of improving motor neuron function of a healthy subject or a subject suffering from impaired motor neuron function by administering an effective amount for improving motor neuron function of a cyclohexanehexyl compound, a pharmaceutically acceptable salt thereof, or a medicament comprising a cyclohexanehexyl compound and a pharmaceutically acceptable carrier, excipient, or vehicle.

In an aspect, a method is provided for treating a mammal in need of improved neuron function, wherein the mammal has no diagnosed disease, disorder, infirmity or ailment known to impair or otherwise diminish neuron function, comprising the step of administering to the mammal a therapeutically effective amount for improving neuron function of a cyclohexanehexyl compound, a pharmaceutically acceptable salt thereof, or a dietary supplement comprising a cyclohexanehexyl compound, or a nutraceutically acceptable derivative thereof.

In an embodiment, the invention relates to a method of slowing degeneration and/or death of neurons in a subject suffering from a polyglutamine disease comprising administering a therapeutically effective amount for slowing degeneration and death of neurons of a cyclohexanehexyl compound, a pharmaceutically acceptable salt thereof, or a medicament comprising a cyclohexanehexyl compound and a pharmaceutically acceptable carrier, excipient, or vehicle.

In a further aspect, the invention provides a method involving administering to a subject a therapeutically effective amount of a cyclohexanehexyl compound, a pharmaceutically acceptable salt thereof, or a medicament comprising a cyclohexanehexyl compound and a pharmaceutically acceptable carrier, excipient, or vehicle which modulates (e.g. inhibits) PolyQ folding and/or aggregation in neurons.

In a further aspect, the invention provides a method involving administering to a subject a therapeutically effective amount of a cyclohexanehexyl compound, a pharmaceutically acceptable salt thereof, or a medicament comprising a cyclohexanehexyl compound and a pharmaceutically acceptable carrier, excipient, or vehicle which causes dissolution/disruption of pre-existing PolyQ aggregates.

In an aspect, the invention provides a method for preventing or inhibiting assembly or slowing deposition of PolyQ aggregates in a subject comprising administering a therapeutically effective amount for preventing or inhibiting assembly or slowing deposition of PolyQ aggregates of a cyclohexanehexyl compound, a pharmaceutically acceptable salt thereof, or a medicament comprising a cyclohexanehexyl compound and a pharmaceutically acceptable carrier, excipient, or vehicle.

In an embodiment, the invention provides a method of reversing or reducing PolyQ aggregates in a subject after the onset of symptoms of a polyglutamine disease comprising administering to the subject a therapeutically effective amount for reversing or reducing PolyQ aggregates after the onset of symptoms of a polyglutamine disease of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention provides a method for enhancing clearance of PolyQ aggregates in a subject comprising administering a therapeutically effective amount for enhancing clearance of PolyQ aggregates of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention provides a method for ameliorating symptoms or onset of a polyglutamine disease comprising administering a therapeutically effective amount for ameliorating symptoms or onset of a polyglutamine disease of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention provides a method for ameliorating progression of a polyglutamine disease comprising administering a therapeutically effective amount for ameliorating progression of a polyglutamine disease of a cyclohexanehexyl compound, a pharmaceutically acceptable salt thereof, or a medicament comprising a cyclohexanehexyl compound and a pharmaceutically acceptable carrier, excipient, or vehicle.

The invention relates to a method for delaying the onset or progression of motor impairment associated with a polyglutamine disease in a subject comprising administering to the subject a therapeutically effective amount for delaying the onset or progression of motor impairment associated with a polyglutamine disease of a cyclohexanehexyl compound, or a medicament comprising a cyclohexanehexyl compound and a pharmaceutically acceptable carrier, excipient, or vehicle.

In an aspect, the invention relates to a method of delaying the progression of a polyglutamine disease comprising administering a therapeutically effective amount for delaying progression of a polyglutamine disease of a cyclohexanehexyl compound, a pharmaceutically acceptable salt thereof, or a medicament comprising a cyclohexanehexyl compound and a pharmaceutically acceptable carrier, excipient, or vehicle.

The invention also relates to a method of increasing survival of a subject suffering from a polyglutamine disease comprising administering a therapeutically effective amount for increasing survival of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention relates to a method of improving the lifespan of a subject suffering from a polyglutamine disease comprising administering a therapeutically effective amount for improving the lifespan of a subject suffering from a polyglutamine disease of a cyclohexanehexyl compound, a pharmaceutically acceptable salt thereof, or a medicament comprising a cyclohexanehexyl compound and a pharmaceutically acceptable carrier, excipient, or vehicle.

In an aspect, the invention relates to a method of preventing a polyglutamine disease in a subject comprising administering a prophylactically effective amount of a cyclohexanehexyl compound, a pharmaceutically acceptable salt thereof, or a medicament comprising a prophylactically effective amount of a cyclohexanehexyl compound and a pharmaceutically acceptable carrier, excipient, or vehicle.

In an aspect, the invention provides a method for protecting neural cells or preventing neuronal death in a subject having a polyglutamine disease comprising administering a prophylactically effective amount of a cyclohexanehexyl compound, a pharmaceutically acceptable salt thereof, or a medicament comprising a prophylactically effective amount of a cyclohexanehexyl compound and a pharmaceutically acceptable carrier, excipient, or vehicle.

In an aspect, the invention relates to a method for delaying the onset or progression of motor impairment associated with a polyglutamine disease in a subject comprising administering a therapeutically effective amount for delaying the onset or progression of motor impairment associated with a polyglutamine disease of a cyclohexanehexyl compound or a medicament comprising a cyclohexanehexyl compound and a pharmaceutically acceptable carrier, excipient, or vehicle.

In an aspect, the invention provides a method for administering a cyclohexanehexyl compound or a medicament comprising a cyclohexanehexyl compound and a pharmaceutically acceptable carrier, excipient, or vehicle in a therapeutically effective amount to patients who need polyglutamine disease treatments while minimizing the occurrence of adverse effects.

In an aspect, the invention provides medicaments for prevention and/or treatment of a polyglutamine disease. Thus, the invention provides a medicament comprising a cyclohexanehexyl compound, in particular a therapeutically effective amount of a cyclohexanehexyl compound for treating a polyglutamine disease. More particularly, the invention provides a medicament in a form adapted for administration to a subject to provide beneficial effects to treat a polyglutamine disease. In an aspect, a medicament is in a form such that administration to a subject suffering from a polyglutamine disease results in modulation of assembly, folding, accumulation, oligomerization, rate of aggregation, oligomerization and/or clearance of proteins or fragments comprising PolyQ, in particular prevention, reduction or inhibition of oligomerization, aggregation and/or assembly of proteins or fragments comprising PolyQ in neurons; reversal or reduction of PolyQ aggregates in neurons after the onset of symptoms of a polyglutamine disease; dissolution and/or disruption of PolyQ aggregates in neurons, and/or enhanced clearance of PolyQ aggregates in neurons; improved neuron function; slowing of degeneration and death of neurons in the brain; increased longevity of a subject; and, slowing or arrest of the progress of a polyglutamine disease.

The invention features a medicament comprising a cyclohexanehexyl compound in a therapeutically effective amount for modulating aggregation or oligomerization of proteins or fragments thereof comprising PolyQ in a subject. In an aspect, the invention provides a medicament comprising a cyclohexanehexyl compound in a therapeutically effective amount for reducing and/or inhibiting aggregation or oligomerization of proteins or fragments thereof comprising PolyQ, or dissolving and/or disrupting pre-existing PolyQ aggregates. The medicament can be in a pharmaceutically acceptable carrier, excipient, or vehicle.

A cyclohexanehexyl compound or medicament comprising a cyclohexanehexyl compound can be administered to a patient by any route effective to treat a polyglutamine disease.

The invention additionally provides a method of preparing a stable medicament comprising one or more cyclohexanehexyl compound in a therapeutically effective amount for treating a polyglutamine disease. After medicaments have been prepared, they can be placed in an appropriate container and labeled for treatment of a polyglutamine disease. For administration of a medicament of the invention, such labeling would include amount, frequency, and method of administration.

The invention also contemplates the use of at least one cyclohexanehexyl compound for treating a polyglutamine disease or in the preparation of a medicament for treating a polyglutamine disease. The invention additionally provides uses of a cyclohexanehexyl for the prevention of a polyglutamine disease or in the preparation of a medicament for the prevention of a polyglutamine disease. A medicament may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder for modulation (e.g., inhibition) of aggregation, oligomerization, formation, deposition, accumulation, clearance and/or persistence of proteins or fragments thereof comprising PolyQ.

The invention further provides a dietary supplement composition comprising one or more cyclohexanehexyl compound or nutraceutically acceptable derivatives thereof, for treatment of a polyglutamine disease, in particular for alleviating the symptoms of a polyglutamine disease. In an aspect, the invention provides a dietary supplement for mammalian consumption and particularly human consumption for the purpose of improving neuron function comprising a cyclohexanehexyl compound, or nutraceutically acceptable derivatives thereof. In another aspect, the invention provides a supplement comprising a cyclohexanehexyl compound, or nutraceutically acceptable derivative thereof for slowing degeneration and death of neurons in the brain of individuals who have taken the supplement and who have a polyglutamine disease or have a predisposition to such a disease. A dietary supplement of the invention is preferably pleasant tasting, effectively absorbed into the body and provides substantial therapeutic effects. In an aspect, a dietary supplement of the present invention is formulated as a beverage, but may be formulated in granule, capsule or suppository form.

The invention also provides a kit comprising one or more cyclohexanehexyl compound, or a medicament comprising same. In an aspect, the invention provides a kit for preventing and/or treating a polyglutamine disease, containing a medicament comprising one or more cyclohexanehexyl compound, a container, and instructions for use. The composition of the kit can further comprise a pharmaceutically acceptable carrier, excipient, or vehicle. In an aspect, the invention provides a method of promoting sales of a medicament or kit of the invention comprising the public distribution of information that administration of the medicament or kit is associated with treatment or prophylaxis of a polyglutamine disease.

These and other aspects, features, and advantages of the present invention should be apparent to those skilled in the art from the following drawings and detailed description.

### DESCRIPTION OF THE DRAWINGS

The invention will be better understood with reference to the drawings in which:

FIG. 1. Characterization of effects of scyllo-inositol on aggregation in inducible PC12 cells expressing a Htt transgene that contains 103 polyglutamine repeats. Upon induction of Htt transgene, soluble poly-Q tagged with EFGP show diffuse fluorescence while aggregated poly-Q demonstrated multiple small fluorescence aggregates with time. In the presence of a known poly-Q aggregation inhibitor, cystamine bitartrate, the presence of aggregated poly-Q is decreased. A concentration dependent effect of decreased aggregation was detected with scyllo-inositol.

FIG. 2. Quantification of aggregation in PC-12 cells expressing Htt103Q-EFGP was determined by counting the number of EGFP positive cells with aggregates in comparison to total EGFP positive cells. Cells were induced to express Htt103Q and either cystamine bitartrate or scyllo-inositol was added simultaneously. * p<0.05, ‡ p<0.001.

FIG. 3. To determine the effect of compounds on protein level of Htt103Q-EFGP, western blot analyses was undertaken. Treatment with cystamine bitartrate decreased aggregation of poly-Q but did not change protein expression levels in inducible PC-12 cells. In contrast, in a concentration dependent manner scyllo-inositol decreased the protein concentration in inducible PC-12 cells.

FIG. 4. Quantification of poly-Q protein expression in inducible PC-12 cells as a function of compound treatment. Cystamine bitratrate and scyllo-inositol up to 10 μM concentration did not alter poly-Q protein expression. At higher concentrations, 25-100 μM, scyllo-inositol decreased poly-Q protein in inducible PC-12 cells. *p<0.05.

### DETAILED DESCRIPTION OF EMBODIMENTS

All technical and scientific terms used herein have the same meaning as commonly understood by one of ordinary skill in the art to which this invention belongs. For convenience, certain terms employed in the specification, examples, and appended claims are collected here.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to “a cyclohexanehexyl compound” includes a mixture of two or more cyclohexanehexyl compounds.

The terms “administering” and “administration” refer to the process by which a therapeutically effective amount of a cyclohexanehexyl compound or medicament contemplated herein is delivered to a subject for prevention and/or treatment purposes. The compounds and medicaments are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disease, or one or more symptoms of such disease, to which such term applies. Treating includes the management and care of a subject at diagnosis or later. A treatment may be either performed in an acute or chronic way. Depending on the condition of the subject, the term may refer to preventing a disease, and includes preventing the onset of a disease, or preventing the symptoms associated with a disease. The term also refers to reducing the severity of a disease or symptoms associated with such disease prior to affliction with the disease. Such prevention or reduction of the severity of a disease prior to affliction refers to administration of a cyclohexanehexyl compound, or medicament comprising same, to a subject that is not at the time of administration afflicted with the disease. “Preventing” also refers to preventing the recurrence of a disease or of one or more symptoms associated with such disease. An objective of treatment is to combat the disease and includes administration of the active compounds to prevent or delay the onset of the symptoms or complications, or alleviating the symptoms or complications, or eliminating or partially eliminating the disease. The terms “treatment” and “therapeutically,” refer to the act of treating, as “treating” is defined above.

The terms “subject”, “individual”, or “patient” are used interchangeably herein and refer to an animal including a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. Among other specific embodiments a mammal of the present invention can be Canis familiaris (dog), Felis catus (cat), Elephas maximus (elephant), Equus caballus (horse), Sus domesticus (pig), Camelus dromedarious (camel), Cervus axis (deer), Giraffa camelopardalis (giraffe), Bos taurus (cattle/cows), Capra hircus (goat), Ovis aries (sheep), Mus musculus (mouse), Lepus brachyurus (rabbit), Mesocricetus auratus (hamster), Cavia porcellus (guinea pig), Meriones unguiculatus (gerbil), or Homo sapiens (human). In a particular embodiment, the mammal is a human. In other embodiments, animals can be treated; the animals can be vertebrates, including both birds and mammals. Birds suitable as subjects within the confines of the present invention include Gallus domesticus (chicken) and Meleagris gallopavo (turkey). Typical subjects for treatment include persons afflicted with or suspected of having or being pre-disposed to a polyglutamine disease, or persons susceptible to, suffering from or that have suffered from a polyglutamine disease. A subject may or may not have a genetic predisposition for a polyglutamine disease. In particular aspects, a subject shows symptoms of a polyglutamine disease. In embodiments of the invention, the subjects are susceptible to, or suffer from a polyglutamine disease.

As utilized herein, the term “healthy subject” means a subject, in particular a mammal, having no diagnosed or symptoms of a polyglutamine disease.

“PolyQ aggregates” refer to aggregates or folded or misfolded proteins, in particular aggregates of proteins associated with polygluatmine diseases or fragments thereof comprising polyQ repeats. The term also includes oligomers of proteins or fragments thereof comprising polyQ repeats. PolyQ aggregates may be intracellular aggregates, neuropil aggregates or they may accumulate in the cytoplasm. Examples of PolyQ aggregates include, without limitation, oligomers, aggregates, and/or folded or misfolded mutant huntingtin (mHtt) protein (HD), mutant dentatorubral-pallidoluysian atrophy or atrophin protein, mutant and truncated androgen receptor protein (spinal and bulbar muscular atrophy/Kennedy's disease), ataxin-1 protein (SCA1), ataxin-3 protein (SCA3/Machado-Joseph disease), CACNA1A (SCA6), ataxin 7 protein (SCAT), and TATA box binding protein (TBP) (SCA17), or parts thereof. (See Table 1 in Paulson H et al., 2000, PNAS 979240: 12957-12958 for a list of mutant proteins and the CAG repeat size in polyglutamine diseases.)

A “beneficial effect” refers to an effect of a cyclohexanehexyl compound or medicament thereof in aspects of the invention, including favorable pharmacological and/or therapeutic effects, and improved biological activity. In aspects of the invention, the beneficial effects include modulation (e.g., inhibition, reversal, or reduction) of assembly, folding, accumulation, oligomerization, rate of aggregation, oligomerization and/or clearance of proteins or fragments comprising PolyQ repeats, in particular prevention, reduction or inhibition of oligomerization, aggregation and/or assembly of proteins or fragments comprising PolyQ repeats in neurons; reversal or reduction of PolyQ aggregates in neurons after the onset of symptoms of a polyglutamine disease, dissolution and/or disruption of PolyQ aggregates in neurons, and/or enhanced clearance of PolyQ aggregates in neurons; improved neuron function; slowing of degeneration and death of neurons in the brain; increased longevity of a subject; and, slowing or arrest of the progress of a polyglutamine disease. In particular aspects of the invention, the beneficial effects include but are not limited to the following: improved motor neuron function, slowing of degeneration and death of neurons in the brain, increased longevity of a subject, and slowing or arrest of the progress of a polyglutamine disease.

In an embodiment, the beneficial effect is a “sustained beneficial effect” where the beneficial effect is sustained for a prolonged period of time after termination of treatment. A treatment can be sustained over several weeks, months or years thereby having a major beneficial impact on the severity of the disease and its complications. In aspects of the invention, a beneficial effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously.

The beneficial effect may be a statistically significant effect in terms of statistical analysis of an effect of a cyclohexanehexyl compound, versus the effects without such a compound. “Statistically significant” or “significantly different” effects or levels may represent levels that are higher or lower than a standard. In embodiments of the invention, the difference may be 1.5, 2, 3, 4, 5, 6, 7, 8, 9, 10, 15, 20, 25, 50, 1-10, 1-20, 1-30 or 1-50 times higher or lower compared with the effect obtained without a cyclohexanehexyl compound.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular medicament. Examples of carriers etc. include but are not limited to saline, buffered saline, dextrose, water, glycerol, ethanol, and combinations thereof. The use of such media and agents for an active substance is well known in the art. Acceptable carriers, excipients or vehicles may be selected from any of those commercially used in the art.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

“Therapeutically effective amount” relates to the amount or dose of an active cyclohexanehexyl compound or medicament thereof, that will lead to one or more desired effects, in particular, one or more beneficial effects. A therapeutically effective amount of a substance can vary according to factors such as the disease state, age, sex, and weight of the subject, and the ability of the substance to elicit a desired response in the subject. A dosage regimen may be adjusted to provide the optimum therapeutic response (e.g. beneficial effects, more particularly sustained beneficial effects). For example, several divided doses may be administered daily or the dose may be proportionally reduced as indicated by the exigencies of the therapeutic situation.

The term “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “pure” in general means better than 90%, 92%, 93%, 94%, 95%, 96%, 97%, 98% or 99% pure, and “substantially pure” means a compound synthesized such that the compound, as made available for consideration into a method or medicament of the invention, has only those impurities that can not readily nor reasonably be removed by conventional purification processes.

As used herein “nutraceutically acceptable derivative” refers to a derivative or substitute for the stated chemical species that operates in a similar manner to produce the intended effect, and is structurally similar and physiologically compatible. Examples of substitutes include without limitation salts, esters, hydrates, or complexes of the stated chemical. The substitute could also be a precursor or prodrug to the stated chemical, which subsequently undergoes a reaction in vivo to yield the stated chemical or a substitute thereof.

“Optional” or “optionally” means that the subsequently described event or circumstance may but need not occur, and that the description includes instances where the event or circumstance occurs and instances in which it does not occur. For example, “alkyl group optionally substituted with a halo group” means that the halo may but need not be present, and the description includes situations where the alkyl group is substituted with a halo group and situations where the alkyl group is not substituted with the halo group.

A “cyclohexanehexyl compound” is understood to refer to any compound, which fully or partially, directly or indirectly, provides one or more therapeutic effects, in particular beneficial effects described herein, and includes a compound of the formula I, II, III or IV described herein, or an analog or derivative thereof (e.g. functional derivative, chemical derivative or variant), salt (e.g., pharmaceutically acceptable salt), prodrug, polymorph, crystalline form, solvate or hydrate thereof. In aspects of the invention, the cyclohexanehexyl compound is an inositol.

A cyclohexanehexyl compound includes a functional derivative, a chemical derivative, or variant. A “functional derivative” refers to a compound that possesses an activity (either functional or structural) that is substantially similar to the activity of a cyclohexanehexyl compound disclosed herein. The term “chemical derivative” describes a molecule that contains additional chemical moieties which are not normally a part of the base molecule. The term “variant” is meant to refer to a molecule substantially similar in structure and function to a cyclohexanehexyl compound or a part thereof. A molecule is “substantially similar” to a cyclohexanehexyl compound if both molecules have substantially similar structures or if both molecules possess similar biological activity. The term “analog” includes a molecule substantially similar in function to a cyclohexanehexyl compound. An “analog” can include a chemical compound that is structurally similar to another but differs slightly in composition. Differences include without limitation the replacement of an atom or functional group with an atom or functional group of a different element. Analogs and derivatives may be identified using computational methods with commercially available computer modeling programs.

A cyclohexanehexyl compound includes a pharmaceutically functional derivative. A “pharmaceutically functional derivative” includes any pharmaceutically acceptable derivative of a cyclohexanehexyl compound, for example, an ester or an amide, which upon administration to a subject is capable of providing (directly or indirectly) a cyclohexanehexyl compound or an active metabolite or residue thereof. Such derivatives are recognizable to those skilled in the art, without undue experimentation (see for example Burger's Medicinal Chemistry and Drug Discovery, 5.sup.th Edition, Vol 1: Principles and Practice, which has illustrative pharmaceutically functional derivatives).

A cyclohexanehexyl compound includes crystalline forms which may exist as polymorphs. Solvates of the compounds formed with water or common organic solvents are also intended to be encompassed within the term. In addition, hydrate forms of the compounds and their salts are encompassed within this invention. Further prodrugs of compounds of cyclohexanehexyl compounds are encompassed within the term.

The term “solvate” means a physical association of a compound with one or more solvent molecules or a complex of variable stoichiometry formed by a solute (for example, a compound of the invention) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. Dehydrate, co-crystals, anhydrous, or amorphous forms of the cyclohexanehexyl compounds are also included. The term “hydrate” means a solvate wherein the solvent molecule(s) is/are H₂O, including, mono-, di-, and various poly-hydrates thereof. Solvates can be formed using various methods known in the art.

Crystalline cyclohexanehexyl compounds can be in the form of a free base, a salt, or a co-crystal. Free base compounds can be crystallized in the presence of an appropriate solvent in order to form a solvate. Acid salt cyclohexanehexyl compounds (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a cyclohexanehexyl compound would have about 1 equivalent of solvent (H₂O) for each equivalent of a cyclohexanehexyl compound. However, more or less solvent may be used depending on the choice of solvate desired.

The cyclohexanehexyl compounds used in the invention may be amorphous or may have different crystalline polymorphs, possibly existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described, for example, in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987, each of which is incorporated herein by reference in their entireties.

Examples of prodrugs include, but are not limited to esters (e.g., acetate, formate, and benzoate derivatives) and carbamates (e.g. N,N-dimethylaminocarbonyl) of hydroxy functional groups on cyclohexanehexyl compounds, and the like

In general, all physical forms of cyclohexanehexyl compounds are intended to be within the scope of the present invention.

In aspects of the invention, the cyclohexanehexyl compound includes a compound with the base structure of the formula I, in particular a substantially pure, compound of the formula I

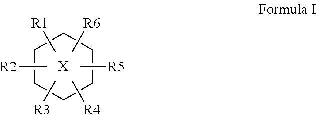

wherein X is a cyclohexane, in particular a myo-, scyllo, epi-, chiro, or allo-inositol radical, wherein one or more of R¹, R², R³, R⁴, R⁵, and R⁶ are independently hydroxyl, alkyl, alkenyl, alkynyl, alkylene, alkenylene, alkoxy, alkenyloxy, cycloalkyl, cycloalkenyl, cycloalkoxy, cycloalkynyl, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and a pharmaceutically acceptable salt, isomer, solvate, or prodrug thereof. In aspects of the invention, four or five or all of R¹, R², R³, R⁴, R⁵, and/or R⁶ are hydroxyl. In particular aspects of the invention, a cyclohexanehexyl compound of the formula I is used wherein X is a radical of scyllo-inositol or epi-inositol.

In an aspect of the invention, a compound of the formula I is utilized wherein X is a cyclohexane, in particular a myo-, scyllo, epi-, chiro, or allo-inositol radical, preferably a scyllo- or epi-inositol radical wherein R¹, R², R³, R⁴, R⁵, and R⁶ are hydroxyl or one or more of R¹, R², R³, R⁴, R⁵, and R⁶ are independently hydroxyl, alkyl, alkenyl, alkynyl, alkylene, alkenylene, alkoxy, alkenyloxy, cycloalkyl, cycloalkenyl, cycloalkoxy, cycloalkynyl, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and the other of R¹, R², R³, R⁴, R⁵, and R⁶ are hydroxyl, or a pharmaceutically acceptable salt, isomer, solvate, or prodrug thereof. In aspects of the invention, four or five or all of R¹, R², R³, R⁴, R⁵, and/or R⁶ are hydroxyl.

Aspects of the invention use classes of cyclohexanehexyl compounds of the formula II, in particular isolated and pure, in particular substantially pure, compounds of the formula II:

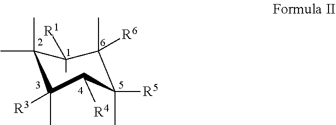

wherein R¹, R², R³, R⁴, R⁵, and R⁶ are hydroxyl, or one or more of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ are hydroxyl, or a pharmaceutically acceptable salt thereof.

In aspects of the invention, the cyclohexanehexyl compound is a substantially pure, compound of the formula I or II as defined herein with the proviso that when (a) one of R¹, R², R³, R⁴, R⁵, and/or R⁶ are alkyl or fluorine no more than four of the other of R¹, R², R³, R⁴, R⁵, and/or R⁶ are hydroxyl, (b) one of R¹, R², R³, R⁴, R⁵, and/or R⁶ is amino or azide no more than four of R¹, R², R³, R⁴, R⁵, and R⁶ are hydroxyl, (c) two of R¹, R², R³, R⁴, R⁵, and/or R⁶ are amino, no more than three of R¹, R², R³, R⁴, R⁵, and R⁶ are hydroxyl, and (d) three of R¹, R², R³, R⁴, R⁵, and/or R⁶ are amino, carboxyl, carbamyl, sulfonyl, isoxasolyl, imidazolyl, or thiazolyl, the other of R¹, R², R³, R⁴, R⁵, and/or R⁶ cannot all be hydroxyl.

In aspects of the invention, the cyclohexanehexyl compound is a substantially pure, compound of the formula III,

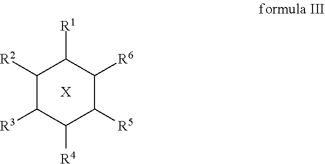

wherein X is a cyclohexane ring, where R¹, R², R³, R⁴, R⁵, and R⁶ are hydroxyl, or at least one of R¹, R², R³, R⁴, R⁵, and R⁶ is independently selected from hydrogen, C₁-C₆ alkyl, C₂-C₆ alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷, —NR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof. In particular aspects the invention utilizes isomers of the compound of the formula III, more particularly scyllo- or epi-isomers.

In aspects of the invention, the cyclohexanehexyl compound is a substantially pure, compound of the formula IV,

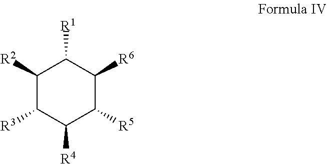

wherein R¹, R², R³, R⁴, R⁵, and R⁶ are defined as for formula III, or a pharmaceutically acceptable salt thereof.

The terms used herein for radicals including “alkyl”, “alkoxy”, “alkenyl”, “alkynyl”, “hydroxyl” etc, refer to optionally substituted radicals, i.e, both unsubstituted and substituted radicals. The term “substituted,” as used herein, means that any one or more moiety on a designated atom (e.g., hydroxyl) is replaced with a selected group provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

“Alkyl”, either alone or within other terms such as “arylalkyl” means a monovalent, saturated hydrocarbon radical which may be a straight chain (i.e. linear) or a branched chain. In certain aspects of the invention, an alkyl radical comprises from about 1 to 24 or 1 to 20 carbon atoms, preferably from about 1 to 10, 1 to 8, 3 to 8, 1 to 6, or 1 to 3 carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents at positions that do not significantly interfere with the preparation of the cyclohexanehexyl compounds and do not significantly reduce the efficacy of the compounds. An alkyl radical may be optionally substituted. In certain aspects, an alkyl radical is substituted with one to five substituents including halo, lower alkoxy, haloalkoxy, alkylalkoxy, haloalkoxyalkyl, hydroxyl, cyano, nitro, thio, amino, substituted amino, carboxyl, sulfonyl, sulfenyl, sulfinyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, aryl (e.g., phenylmethyl (i.e. benzyl)), heteroaryl (e.g., pyridyl), and heterocyclic (e.g., piperidinyl, morpholinyl).

In aspects of the invention, “substituted alkyl” refers to an alkyl group substituted by, for example, one to five substituents, and preferably 1 to 3 substituents, such as alkyl, alkoxy, oxo, alkanoyl, aryl, aralkyl, aryloxy, alkanoyloxy, cycloalkyl, acyl, amino, hydroxyamino, alkylamino, arylamino, alkoxyamino, aralkylamino, cyano, halogen, hydroxyl, carboxyl, carbamyl, carboxylalkyl, keto, thioketo, thiol, alkylthiol, arylthio, aralkylthio, sulfonamide, thioalkoxy, and nitro.

The term “alkenyl” refers to an unsaturated, acyclic branched or straight-chain hydrocarbon radical comprising at least one double bond. Alkenyl radicals may contain from about 2 to 24 or 2 to 10 carbon atoms, preferably from about 3 to 8 carbon atoms and more preferably about 3 to 6 or 2 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. Preferred alkenyl groups include ethenyl (—CH═CH₂), n-propenyl (—CH₂CH═CH₂), iso-propenyl (—C(CH₃)═CH₂), and the like. An alkenyl radical may be optionally substituted similar to alkyl.

In aspects of the invention, “substituted alkenyl” refers to an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkynyl” refers to an unsaturated, branched or straight-chain hydrocarbon radical comprising one or more triple bonds. Alkynyl radicals may contain about 1 to 20, 1 to 15, or 2-10 carbon atoms, preferably about 3 to 8 carbon atoms and more preferably about 3 to 6 carbon atoms. In aspects of the invention, “alkynyl” refers to straight or branched chain hydrocarbon groups of 2 to 6 carbon atoms having one to four triple bonds. Examples of suitable alkynyl radicals include ethynyl, propynyls, such as prop-1-yn-1-yl, prop-2-yn-1-yl, butynyls such as but-1-yn-1-yl, but-1-yn-3-yl, and but-3-yn-1-yl, pentynyls such as pentyn-1-yl, pentyn-2-yl, and 4-methoxypentyn-2-yl, and 3-methylbutyn-1-yl, hexynyls such as hexyn-1-yl, hexyn-2-yl, and hexyn-3-yl, and 3,3-dimethylbutyn-1-yl radicals and the like. This radical may be optionally substituted similar to alkyl. The term “cycloalkynyl” refers to cyclic alkynyl groups.

In aspects of the invention, “substituted alkynyl” refers to an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, ethylidene, methylethylene, and isopropylidene.

The term “alkenylene” refers to a linear or branched radical having from about 2 to 10, 2 to 8 or 2 to 6 carbon atoms, at least one double bond, and having attachment points for two or more covalent bonds. Examples of such radicals are 1,1-vinylidene (CH₂═C), 1,2-vinylidene (—CH═CH—), and 1,4-butadienyl (—CH═CH—CH═CH—).

As used herein, “halogen” or “halo” refers to fluoro, chloro, bromo and iodo, especially fluoro or chloro.

The term “hydroxyl” or “hydroxy” refers to a single —OH group.

The term “cyano” refers to a carbon radical having three of four covalent bonds shared by a nitrogen atom, in particular —CN.

The term “alkoxy” refers to a linear or branched oxy-containing radical having an alkyl portion of one to about ten carbon atoms, which may be substituted. Particular alkoxy radicals are “lower alkoxy” radicals having about 1 to 6, 1 to 4 or 1 to 3 carbon atoms. An alkoxy having about 1-6 carbon atoms includes a C₁-C₆ alkyl-O— radical wherein C₁-C₆ alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g., alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

In aspects of the invention, “acyl” refers to a group —C(O)R⁹, where R⁹ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

The term “cycloalkyl” refers to radicals having from about 3 to 16 or 3 to 15 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃-C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted.

In aspects of the invention, “substituted cycloalkyl” refers to cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

The term “cycloalkenyl” refers to radicals comprising about 2 to 16, 4 to 16, 2 to 15, 2 to 10, 4 to 10, 3 to 8, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms, in particular cyclobutenyl, cyclopentenyl, cyclohexenyl and cycloheptenyl. A cycloalkenyl radical may be optionally substituted with groups as disclosed herein.

The term “cycloalkoxy” refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. In aspects of the invention an aryl radical comprises 4 to 24 carbon atoms, in particular 4 to 10, 4 to 8, or 4 to 6 carbon atoms. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, diphenyl, acephthylenyl, fluorenyl, phenalenyl, phenanthrenyl, and anthracenyl, preferably phenyl. An aryl radical may be optionally subsitituted (“substituted aryl”), for example, with one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenyzl, and amino benzyl.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy” as used herein, refers to an aryl group attached to an alkoxy group. Representative examples of arylalkoxy include, but are not limited to, 2-phenylethoxy, 3-naphth-2-ylpropoxy, and 5-phenylpentyloxy.

The term “aroyl” refers to aryl radicals, as defined above, attached to a carbonyl radical as defined herein, including without limitation benzoyl and toluoyl. An aroyl radical may be optionally substituted with groups as disclosed herein.

The term “heteroaryl” refers to fully unsaturated heteroatom-containing ring-shaped aromatic radicals having from 3 to 15, 3 to 10, 5 to 15, 5 to 10, or 5 to 8 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed herein.

The term “heterocyclic” refers to saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitiation a saturated 3 to 6-membered heteromonocylic group containing 1 to 4 nitrogen atoms [e.g. pyrrolidinyl, imidazolidinyl, piperidinyl, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like.

The term “sulfate”, used alone or linked to other terms, is art recognized and includes a group that can be represented by the formula:

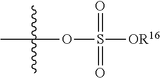

wherein R¹⁶ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfonyl”, used alone or linked to other terms such as alkylsulfonyl or arylsulfonyl, refers to the divalent radicals —SO₂—. In aspects of the invention where one or more of R¹, R³, R⁴, R⁵, or R⁶ is a sulfonyl group, the sulfonyl group may be attached to a substituted or unsubstituted alkyl group, alkenyl group, alkynyl group, aryl group, cycloalkyl group, cycloalkenyl group, cycloalkynyl group, or heterocyclic group, carbohydrate, peptide, or peptide derivative.

The term “sulfonate” is art recognized and includes a group represented by the formula:

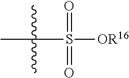

wherein R¹⁶ is an electron pair, hydrogen, alkyl, cycloalkyl, aryl, alkenyl, alkynyl, cycloalkenyl, cycloalkynyl, heterocyclic, carbohydrate, peptide, or peptide derivative

Examples of sulfonated alkyl groups include ethyl sulfuric acid, ethanesulfonic acid, 2-aminoethan-1-ol sulfuric acid, 1-propanesulfonic acid, 2-propanesulfonic acid, 1,2-diethanedisulfonic acid, 1,2-ethanediol disulfuric acid, 1,3-propanedisulfonic acid, 1-propanol sulfuric acid, 1,3-propanediol disulfuric acid, 1-butanesulfonic acid, 1,4-butanediol disulfuric acid, 1,2-ethanediol disulfuric acid, 3-amino-1-propanesulfonic acid, 3-hydroxypropanesulfonic acid sulfate, 1,4-butanesulfonic acid, 1,4-butanediol monosulfuric acid, 1-pentanesulfonic acid, 1,5-pentanedisulfonic acid, 1,5-pentanediol sulfuric acid, 4-heptanesulfonic acid, 1,3,5-heptanetriol trisulfate, 2-hydroxymethyl-1,3-propanediol trisulfate, 2-hydroxymethyl-2-methyl-1,3-propanediol trisulfate, 1,3,5,7-heptanetetraol tetrasulfate, 1,3,5,7,9-nonane pentasulfate, 1-decanesulfonic acid, and pharmaceutically acceptable salts thereof.

Examples of cycloalkyl sulfonated groups include 1,3-cyclohexanediol disulfate, and 1,3,5-heptanetriol trisulfate.

Examples of aryl sulfonated groups include 1,3-benzenedisulfonic acid, 2,5-dimethoxy-1,4-benzenedisulfonic acid, 4-amino-3-hydroxy-1-naphthalenesulfonic acid, 3,4-diamino-1-naphthalenesulfonic acid, and pharmaceutically acceptable salts thereof.

Examples of heterocyclic sulfonated compounds include 3-(N-morpholino)propanesulfonic acid and tetrahydrothiophene-1,1-dioxide-3,4-disulfonic acid, and pharmaceutically acceptable salts thereof.

Examples of sulfonated carbohydrates are sucrose octasulfonate, 5-deoxy-1,2-O-isopropylidene-α-D-xylofuranose-5-sulfonic acid or an alkali earth metal salt thereof, methyl-α-D-glucopyranoside 2,3-disulfate, methyl 4, —O-benzylidene-α-D-glucopyranoside 2,3-disulfate, 2,3,4,3′,4′-sucrose pentasulfate, 1,3:4,6-di-O-benzylidene-D-mannitol 2,5-disulfate, D-mannitol 2,5-disulfate, 2,5-di-O-benzyl-D-mannitol tetrasulfate, and pharmaceutically acceptable salts thereof.

The term “sulfinyl”, used alone or linked to other terms such as alkylsulfinyl (i.e. —S(O)-alkyl) or arylsulfinyl, refers to the divalent radicals —S(O)—.

The term “sulfoxide” refers to the radical —S═O.

The term “amino”, alone or in combination, refers to a radical where a nitrogen atom (N) is bonded to three substituents being any combination of hydrogen, hydroxyl, alkyl, cycloalkyl, alkenyl, alkynyl, aryl or silyl with the general chemical formula —NR¹⁰R¹¹ where R¹⁰ and R¹¹ can be any combination of hydrogen, hydroxyl, alkyl, cycloalkyl, alkenyl, alkynyl, aryl, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidine, benzylamino, diphenylmethylamino, tritylamino, trimethylsilylamino, and dimethyl-tert.-butylsilylamino.

The term “thiol” means —SH.

The term “sulfenyl” refers to the radical —SR¹² wherein R¹² is not hydrogen. R¹² may be alkyl, alkenyl, alkynyl, cycloalkyl, aryl, silyl, heterocyclic, heteroaryl, carbonyl, or carboxyl.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR¹³ where R¹³ is an aryl group which may be substituted. Illustrative examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “thioalkoxy”, alone or in combination, refers to a chemical functional group where a sulfur atom (S) is bonded to an alkoxy group with the general chemical formula —SR¹⁵ where R¹⁵ is an alkoxy group which may be substituted. In aspects of the invention a “thioalkoxy group” has 1-6 carbon atoms and refers to a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

The term “carbonyl” refers to a carbon radical having two of the four covalent bonds shared with an oxygen atom.

The term “carboxyl”, alone or in combination, refers to —C(O)OR¹⁴— wherein R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted. In aspects of the invention, the carboxyl groups are in an esterified form and may contain as an esterifying group lower alkyl groups. In particular aspects of the invention, —C(O)OR¹⁴ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amine, halo, alkylamino, aryl, carboxyl, or a heterocyclic. In particular aspects of the invention, the carboxyl group is methoxycarbonyl, butoxycarbonyl, tert.alkoxycarbonyl such as tert butoxycarbonyl, arylmethyoxycarbonyl having one or two aryl radicals including without limitation phenyl optionally substituted by, for example, lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyl and dimethyltert.butylsilyl.

The term “carboxamide”, alone or in combination, refers to amino, monoalkylamino, dialkylamino, monocycloalkylamino, alkylcycloalkylamino, and dicycloalkylamino radicals, attached to one of two unshared bonds in a carbonyl group.

The term “nitro” means —NO₂—.

A radical in a cyclohexanehexyl compound may be substituted with one or more substituents apparent to a person skilled in the art including without limitation alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, sulfenyl, alkylsulfonyl, sulfinyl, alkylsulfinyl, aralkyl, heteroaralkyl, cycloalkyl, cycloalkenyl, cycloalkoxy, cycloalkenyloxy, amino, oxy, halo, azido, thio, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfonyl, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. In embodiments of the invention, the substituents include alkyl, alkoxy, alkynyl, halo, amino, thio, oxy, and hydroxyl.

While broad definitions of cyclohexanehexyl compounds are described herein for use in the present invention, certain compounds of formula I, H, III or IV may be more particularly described.

In embodiments of the invention, the cyclohexanehexyl compound is an isolated, in particular pure, more particularly substantially pure, compound of the formula I, wherein X is a radical of scyllo-inositol, epi-inositol or a configuration isomer thereof, wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In embodiments of the invention, the cyclohexanehexyl compound is an isolated, in particular pure, more particularly, substantially pure, compound of the formula II wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In particular aspects of the invention, a cyclohexanehexyl compound does not include a compound of the formula I or II where (a) when one of R¹, R², R³, R⁴, R⁵, and/or R⁶ are alkyl or fluorine, more than 4 of the other of R¹, R², R³, R⁴, R⁵, and/or R⁶ are hydroxyl, (b) when one of R¹, R², R³, R⁴, R⁵, and/or R⁶ is amino or azide, more than four of R¹, R², R³, R⁴, R⁵, and/or R⁶ are hydroxyl, (c) when two of R¹, R², R³, R⁴, R⁵, and/or R⁶ are amino, more than three of R¹, R², R³, R⁴, R⁵, and/or R⁶ are hydroxyl, and (d) R¹, R², R³, R⁴, R⁵, and/or R⁶ are isopropylidene.

In some aspects of the invention, a cyclohexanehexyl compound is utilized where one or more of R¹, R², R³, R⁴, R⁵, and/or R⁶ are alkyl, alkoxy, or halo, and the other of R¹, R², R³, R⁴, R⁵, and/or R⁶ is hydrogen.

In embodiments of the invention, the cyclohexanehexyl compound is a compound of the formula I or II where the hydrogen at one or more of positions 1, 2, 3, 4, 5, or 6 of formula I or II is substituted with a radical disclosed herein for R¹, R², R³, R⁴, R⁵, and R⁶, including optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular optionally substituted alkyl, alkenyl, alkoxy, amino, imino, thiol, nitro, cyano, halo, or carboxyl.

In embodiments of the invention, the cyclohexanehexyl compound is a compound of the formula I or II wherein one or more of, two or more of, or three or more of R¹, R², R³, R⁴, R⁵, and/or R⁶ are independently alkenyl, alkynyl, alkylene, alkenylene, alkoxy, alkenyloxy, cycloalkenyl, cycloalkoxy, aryl, aryloxy, arylalkoxy, aroyl, heteroaryl, heterocyclic, acyl, acyloxy, sulfonyl, sulfenyl, sulfinyl, sulfonate, sulfoxide, sulfate, nitro, cyano, isocyanato, thioaryl, thioalkoxy, seleno, silyl, silyloxy, silylthio, Cl, I, Br, carboxyl, carboxylic ester, carbonyl, carbamoyl, or carboxamide and the other of R¹, R², R³, R⁴, R⁵, and/or R⁶ is a hydroxyl.

In embodiments of the invention, the cyclohexanehexyl compound is an isolated, in particular pure, more particularly, substantially pure, compound of the formula I or II wherein one or more of, two or more of, or three or more of R¹, R², R³, R⁴, R⁵, and/or R⁶ are independently C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, C₃-C₈ cycloalkoxy, acyloxy, sulfonyl, sulfenyl, sulfinyl, sulfonate, sulfoxide, sulfate, isocyanato, thioaryl, thioalkoxy, selene, silyl, silyloxy, silythio, aryl, aroyl, aryloxy, arylC₁-C₆alkoxy, acetyl, heteroaryl, heterocyclic, amino, thiol, thioalkyl, thioalkoxy, nitro, cyano, halo (e.g., Cl, I, or Br), carboxyl, carboxylic ester, carbonyl, carbamoyl, or carboxamide and the other of R¹, R², R³, R⁴, R⁵, and/or R⁶ is a hydroxyl. In particular aspects, (a) when one of R¹, R², R³, R⁴, R⁵, and/or R⁶ are alkyl or fluorine no more than 4 of the other of R¹, R², R³, R⁴, R⁵, and/or R⁶ are hydroxyl, (b) when one of R¹, R², R³, R⁴, R⁵, and/or R⁶ is amino no more than four of R¹, R², R³, R⁴, R⁵, and/or R⁶ are hydroxyl, (c) when two of R¹, R², R³, R⁴, R⁵, and/or R⁶ are amino, no more than three of R¹, R², R³, R⁴, R⁵, and R⁶ are hydroxyl, and (d) R¹, R², R³, R⁴, R⁵, and/or R⁶ are not isopropylidene.

In embodiments of the invention, the cyclohexanehexyl compound is a compound of the formula I wherein R² is hydroxyl in an equatorial position, at least one, two, three, or four of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfenyl, sulfonyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexanehexyl compound is a compound of the formula I wherein R² is hydroxyl in an equatorial position, at least two of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexanehexyl compound is a compound of the formula II wherein R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, thioalkyl, thioalkoxy, thioaryl, nitro, cyano, halo, silyl, silyloxy, carboxyl, carboxylic ester, carbonyl, carbamoyl, or carboxamide and the other of R¹, R³, R⁴, R⁵, and R⁶ is hydroxyl.

In embodiments of the invention, the cyclohexanehexyl compound is a compound of the formula I or II wherein at least two of R¹, R², R³, R⁴, R⁵, and/or R⁶ are hydroxyl, and one, two, three or four or more of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein at least two of R¹, R², R³, R⁴, R⁵, and/or R⁶ are hydroxyl, and two or more of the other of R¹, R², R³, R⁴, R⁵, and/or R⁶ are alkyl, cycloalkyl, alkenyl, cycloalkenyl, alkynyl, alkylene, alkenylene, alkoxy, alkenyloxy, cycloalkoxy, aryl, aryloxy, arylalkoxy, aroyl, heteroaryl, heterocyclic, acyl, or acyloxy, sulfonyl, sulfenyl, sulfinyl, amino, imino, cyano, isocyanato, seleno, silyl, silyloxy, silylthio, thiol, thioalkyl, thioalkoxy, halo, carboxyl, carboxylic ester, carbonyl, carbamoyl, and carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein at least two of R¹, R², R³, R⁴, R⁵, and/or R⁶ are hydroxyl, and three or more of the other of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein at least three of R¹, R², R³, R⁴, R⁵, and/or R⁶ are hydroxyl, and one, two, or three of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein at least four of R¹, R², R³, R⁴, R⁵, and/or R⁶ are hydroxyl, and one or two of the other of R¹, R³, R⁴, R⁵, and/or R⁶ are alkyl, alkenyl, alkynyl, alkylene, to alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfonate, sulfenyl, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein R¹, R², R⁴, R⁵, and R⁶ are hydroxyl, and R³ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R³ is selected from the group consisting of alkenyl, alkynyl, alkylene, alkenylene, alkoxy, alkenyloxy, cycloalkyl, cycloalkenyl, cycloalkoxy, aryl, aryloxy, arylalkoxy, aroyl, imino, heteroaryl, heterocyclic, acyl, acyloxy, sulfonyl, sulfenyl, sulfinyl, sulfoxide, sulfate, thioalkoxy, thioaryl, carboxyl, carbonyl, carbamoyl, or carboxamide, in particular alkoxy, sulfonyl, sulfenyl, sulfinyl, sulfoxide, sulfate, thioalkoxy, carboxyl, carbonyl, carbamoyl, or carboxamide. In a particular embodiment, R³ is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexyl compound is a compound of the formula I or II wherein R¹, R³, R⁴, R⁵, and R⁶ are hydroxyl, and R² is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R² is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester.

In embodiments of the invention, the cyclohexanehexyl compound is a compound of the formula I, II, III or IV wherein one, two, three, four or five of R¹, R², R³, R⁴, R⁵, and/or R⁶ are each independently:
(a) alkyl with 1 to 24 carbon atoms, in particular 1 to 10 or 1 to 6 carbon atoms;
(b) cycloalkyl with 3 to 16 carbon atoms, in particular 3 to 10 or 3 to 6 carbon atoms;
(c) alkenyl with 2 to 24 carbon atoms, in particular 2 to 10 or 2 to 6 carbon atoms;
(d) cycloalkenyl with 4 to 16 carbon atoms, in particular 4 to 10 or 4 to 6 carbon atoms;
(e) aryl with 4 to 24 carbon atoms, in particular 4 to 10, 4 to 8, or 6 or carbon atoms;
(f) aralkyl, alkaryl, aralkenyl, or alkenylaryl;
(g) heterocyclic group comprising 3 to 10, in particular 3 to 8 or 3 to 6 ring members and at least one atom selected from the group consisting of oxygen, nitrogen, and sulfur;
(h) alkoxy with 1 to 6 carbon atoms or 1 to 3 carbon atoms in particular methoxy, ethoxy, propoxy, butoxy, isopropoxy or tert-butoxy, especially methoxy, or
(i) halo, in particular fluorine, chlorine, or bromine, especially chlorine.

In embodiments of the invention, the cyclohexanehexyl compound is a compound of the formula I, II, III or IV wherein R² is hydroxyl and one, two, three, four or five of R¹, R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cyclopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexanehexyl compound is a compound of the formula I, II, III or IV wherein R¹ is hydroxyl and one, two, three, four or five of R², R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cyclopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexanehexyl compound is a compound of the formula I, II, III or IV wherein one or two of R¹, R², R³, R⁴, R⁵, and/or R⁶ are carboxyl, carbamyl, sulfonyl, or a heterocyclic comprising a N atom, more particularly N-methylcarbamyl, N-propylcarbamyl, N-cyanocarbamyl, aminosulfonyl, isoxazolyl, imidazolyl, and thiazolyl.

In embodiments of the invention, a cyclohexanehexyl compound of the formula III or IV is utilized wherein X is a cyclohexane, R¹, R², R³, R⁴, R⁵, and R⁶ are hydroxyl or at least one of R¹, R², R³, R⁴, R⁵, and R⁶ is independently selected from hydrogen, C₁-C₆ alkyl, C₂-C₆ alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷, —NR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof.

In embodiments of the invention, the cyclohexanehexyl compound is a compound of the formula I, II, III or IV where R² is hydroxyl; and R¹, R³, R⁴, R⁵, and R⁶ are independently selected from C₁-C₆alkyl, C₂-C₆alkenyl, C₂-C₆alkynyl, C₁C₆ alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀ heterocyclic, C₁-C₆acyl, C₁-C₆acyloxy, hydroxyl, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀ cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic; provided that R¹, R², R³, R⁴, R⁵, and R⁶ are not all hydroxyl.

In embodiments of the invention, the cyclohexanehexyl compound is a compound of the formula I, II, III or IV where R² is hydroxyl; one of R¹, R³, R⁴, R⁵, and R⁶ is hydroxyl; and four of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexanehexyl compound is a compound of the formula I, II, III or IV where R² is hydroxyl; two of R¹, R³, R⁴, R⁵, and R⁶ are hydroxyl; and three of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexyl compound is a compound of the formula III or IV where R² is hydroxyl; three of R¹, R³, R⁴, R⁵, and R⁶ is hydroxyl; and two of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexyl compound is a compound of the formula III or IV where R² is hydroxyl; four of R¹, R³, R⁴, R⁵, and R⁶ are hydroxyl; and one of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀ aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexyl compound is a compound of the formula III or IV wherein one of R¹, R³, R⁴, R⁵, and R⁶ is C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷ and R⁸ are as defined above; and no more than four of the remainder of R¹, R², R³, R⁴, R⁵, and R⁶ are hydroxyl.

In embodiments of the invention, the cyclohexanehexyl compound is a compound of the formula III or IV wherein two of R¹, R³, R⁴, R⁵, and R⁶ are C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷ and R⁸ are as defined above; and no more than three of R¹, R², R³, R⁴, R⁵, and R⁶ are hydroxyl.

In embodiments of the invention, the cyclohexanehexyl compound is a compound of the formula III or IV wherein three of R¹, R³, R⁴, R⁵, and R⁶ are C₁-C₆alky, C₁-C₆alkoxy, C₁-C₆alkyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷ and R⁸ are as defined above; and no more than two of R¹, R², R³, R⁴, R⁵, and R⁶ are hydroxyl.

In embodiments of the invention, the cyclohexanehexyl compound is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexyl compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexyl compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R²R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexyl compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexyl compound is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexyl compound is a compound of the formula I, II, III or IV wherein one, two, or three of R¹, R², R³, R⁴, R⁵, and/or R⁶ is each independently —OR¹⁷ where R¹⁷ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide or a carbohydrate. In an aspect, wherein one, two, or three of R¹, R², R³, R⁴, R⁵, and/or R⁶ is each independently —OR¹⁷ where R¹⁷ is C₁-C₆ alkyl, most particularly C₁-C₃ alkyl.

In selected cyclohexanehexyl compounds of the formula I, II, III or IV, at least one of R¹, R², R³, R⁴, R⁵, and/or R⁶ is —OR²⁰ wherein R²⁰ is —CF₃, CF₃CF₂, CF₃CH₂, CH₂NO₂, CH₂NH₂, C(CH₂)₃, or cyclopropyl.

In embodiments of the invention, the cyclohexanehexyl compound is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁵ are hydroxyl and R⁶ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁵ are hydroxyl and R⁶ is methoxy.

In embodiments of the invention, the cyclohexanehexyl compound is a compound of the formula I, II, III or IV wherein R¹, R², R³, R⁴, R⁵, and R⁶ are hydroxyl and R⁵ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁶ are hydroxyl and R⁵ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁶ are hydroxyl and R⁵ is methoxy.

In embodiments of the invention, the cyclohexanehexyl compound is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R³, R⁵, and R⁶ are hydroxyl and R⁴ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁵, and R⁶ are hydroxyl and R⁴ is methoxy.

In embodiments of the invention, the cyclohexanehexyl compound is a compound of the formula I, II, III or IV wherein R¹, R², R³, R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R⁴, R⁵, and R⁶ are hydroxyl and R³ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R⁴, R⁵, and R⁶ are hydroxyl and R³ is methoxy.

In embodiments of the invention, the cyclohexanehexyl compound is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R³, R⁴, R⁵, and R⁶ are hydroxyl and R² is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R³, R⁴, R⁵, and R⁶ are hydroxyl and R² is methoxy.

In embodiments of the invention, the cyclohexanehexyl compound is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R², R³, R⁴, R⁵, and R⁶ are hydroxyl and R¹ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R², R³, R⁴, R⁵, and R⁶ are hydroxyl and R¹ is methoxy.

In embodiments of the invention, the cyclohexanehexyl compound is a compound of the formula III or IV, wherein two, three, four or five of R¹, R², R³, R⁴, R⁵, or R⁶ are hydroxyl; at least one of R¹, R², R³, R⁴, or R⁶ is optionally substituted alkoxy; and the remainder of R¹, R², R³, R⁴, R⁵, or R⁶ if any are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆acyl, C₁-C₆ acyloxy, hydroxyl, —NH₂, —NHR⁷, —NR⁷R⁸—, —S(O)₂R⁷, —SH, nitro, cyano, halo, haloalkyl, haloalkoxy, hydroxyalkyl, —CO₂R⁷, oxo, —PO₃H —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀arylC₁-C₃alkyl, C₆-C₁₀heteroaryl and C₃-C₁₀heterocyclic.

In embodiments of the invention, the cyclohexanehexyl compound is a compound of the formula III or IV, wherein five of R¹, R², R³, R⁴, R⁵, or R⁶ are hydroxyl; and one of R¹, R², R³, R⁴, R⁵, or R⁶ is C₁-C₆alkoxy; for example at least one of R¹, R², R³, R⁴, R⁵, or R⁶ is methoxy.

In embodiments of the invention, the cyclohexanehexyl compound is a compound of the formula IV, wherein two, three, or four of R², R³, R⁴, R⁵, or R⁶ are hydroxyl; R¹ is optionally substituted alkoxy; and the remainder of R², R³, R⁴, R⁵, or R⁶ are independently selected from C₁-C₆alkyl, C₂-C₆alkenyl, C₂-C₆alkynyl, C₁-C₆alkoxy, C₂-C₆alkenyloxy, C₃-C₁₀cycloalkyl, C₁-C₆acyl, C₁-C₆acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexanehexyl compound is a compound of the formula IV, wherein R¹ is C₁-C₆ alkoxy; and R², R³, R⁴, R⁵, and R⁶ are hydroxyl; for example R¹ is methoxy.

In embodiments of the invention, the cyclohexanehexyl compound is a compound of the formula I, II, III or IV wherein five of R¹, R⁴, R⁵, and/or R⁶ are hydroxyl and the other of R¹, R², R³, R⁴, R⁵, and/or R⁶ is substituted alkoxy, in particular alkoxy having about 1-6 carbon atoms, more particularly methoxy, ethoxy, propoxy, butoxy, isopropoxy and tert-butoxy, substituted with alkyl, in particular C₁-C₆ alkyl, more particularly C₁-C₃ alkyl.

In embodiments of the invention, the cyclohexanehexyl compound is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy substituted with halo (e.g., fluoro, chloro or bromo) which may be substituted. In particular embodiments five of R¹, R², R³, R⁴, R⁵, and/or R⁶ are hydroxyl and the other of R¹, R², R³, R⁴, R⁵, and/or R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein five of R¹, R², R³, R⁴, R⁵, and/or R⁶ are hydroxyl and the other of R¹, R², R³, R⁴, R⁵, and/or R⁶ is a haloalkoxyalkyl, in particular fluoromethoxymethyl, chloromethoxyethyl, trifluoromethoxymethyl, difluoromethoxyethyl, or trifluoroethoxymethyl.

In embodiments of the invention, the cyclohexanehexyl compound is a compound of the formula I, II, III or IV wherein R¹, R², R³, R⁴, and R⁵ are hydroxyl and R⁶ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, the cyclohexanehexyl compound is a compound of the formula I, II, III or IV wherein R¹, R², R³, R⁴, and R⁶ are hydroxyl and R⁵ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexyl compound is a compound of the formula I, II, III or IV wherein R¹, R², R³, R⁵, and R⁶ are hydroxyl and R⁴ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexyl compound is a compound of the formula I, II, III or IV wherein R¹, R², R⁴, R⁵, and R⁶ are hydroxyl and R³ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexyl compound is a compound of the formula I, II, III or IV wherein R¹, R³, R⁴, R⁵, and R⁶ are hydroxyl and R² is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexyl compound is a compound of the formula I, II, III or IV wherein R², R³, R⁴, R⁵, and R⁶ are hydroxyl and R¹ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexyl compound is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁵ are hydroxyl and R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of R², the formula I, II, III or IV wherein R¹, R², R³, R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, R⁵, and R⁶ are hydroxyl and R⁵ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R³, R⁴, R⁵, and R⁶ are hydroxyl and R² is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R², R³, R⁴, R⁵, and R⁶ are hydroxyl and R¹ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexyl compound is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester. In aspects of the invention at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexyl compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexyl compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexyl compound is a compound of the formula I, II, III or IV wherein five of R¹, R², R³, R⁴, R⁵, or R⁶ are hydroxyl and the other of R¹, R², R³, R⁴, R⁵, or R⁶ is a carboxylic ester.

In embodiments of the invention, the cyclohexanehexyl compound is a compound of the formula I, II, III or IV wherein at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R¹, R², R³, R⁴, and R⁵ are hydroxyl and R⁶ is a carboxylic ester. In aspects of the invention, R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R¹, R², R³, R⁴, and R⁶ are hydroxyl and R⁵ is a carboxylic ester. In aspects of the invention, R⁵ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R¹, R², R³, R⁵, and R⁶ are hydroxyl and R⁴ is a carboxylic ester. In aspects of the invention, R⁴ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R¹, R², R³, R⁴, R⁵, and R⁶ are hydroxyl and R³ is a carboxylic ester. In aspects of the invention, R³ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R¹, R³, R⁴, R⁵, and R⁶ are hydroxyl and R² is a carboxylic ester. In aspects of the invention, R² is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexyl compound is a compound of the formula I, II, III or IV wherein R², R³, R⁴, R⁵, and R⁶ are hydroxyl and R¹ is a carboxylic ester. In aspects of the invention, R¹ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic. In particular embodiments, R¹⁴ is selected to provide an amino acid derivative or an ester derivative. In preferred embodiments of the invention R¹⁴ is one of the following:

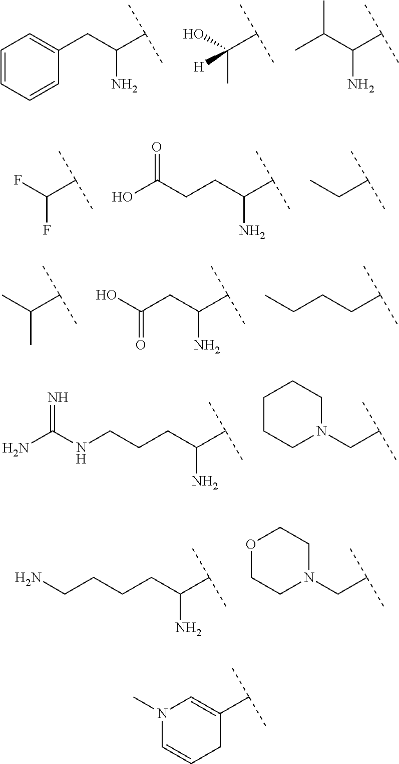

In embodiments of the invention, the cyclohexanehexyl compound is a compound of the formula I, II, III or IV wherein one, two or three of R¹, R², R³, R⁴, R⁵, and/or R⁶ is each independently:

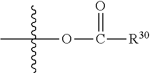

where R³⁰ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and the other of R¹, R², R³, R⁴, R⁵, and/or R⁶ is hydroxyl.

In embodiments of the invention, the cyclohexanehexyl compound is a compound of the formula I, II, III or IV wherein at least one, two, three or four of R¹, R³, R⁴, R⁵, and/or R⁶ are hydroxyl and the other of R¹, R³, R⁴, R⁵, and/or R⁶ are alkyl, halo, alkoxy, sulfonyl, sulfinyl, thiol, thioalkyl, thioalkoxy, carboxyl, in particular C₁-C₆ alkyl, C₁-C₆ alkoxy, or halo.

In embodiments of the invention, the cyclohexanehexyl compound is a compound of the formula I, II, III or IV wherein R¹, R², R³, R⁴, R⁵, and/or R⁶ is each independently —CH₃, —OCH₃, F, N₃, NH₂, SH, NO₂, CF₃, OCF₃, SeH, Cl, Br, I or CN with the proviso that four or five of R¹, R², R³, R⁴, R⁵, and/or R⁶ are hydroxyl.

In embodiments of the invention, the cyclohexanehexyl compound is a compound of the formula I, H, III or IV wherein five of R¹, R², R³, R⁴, R⁵, and/or R⁶ are hydroxyl and one of R¹, R², R³, R⁴, R⁵, or R⁶, and more particularly R² or R³, is selected from the group consisting of —CH₃, —OCH₃, CF₃, F, SeH, Cl, Br, I and CN.

In embodiments of the invention, the cyclohexanehexyl compound is a compound of the formula I, II, III or IV wherein four of R¹, R², R³, R⁴, R⁵, and/or R⁶ are hydroxyl and two of R¹, R², R³, R⁴, R⁵, and/or R⁶ are selected from the group consisting of —CH₃, —OCH₃, CF₃, F, —NO₂, SH, SeH, Cl, Br, I and CN.

In embodiments of the invention, the cyclohexanehexyl compound is a compound of the formula III or IV, wherein four of R¹, R², R³, R⁴, R⁵, or R⁶ are hydroxyl; and one of R¹, R², R³, R⁴, R⁵, or R⁶ is each independently selected from the group CH₃, OCH₃, NO₂, CF₃, OCF₃, F, Cl, Br, I and CN.

In embodiments of the invention, the cyclohexanehexyl compound is a compound of the formula III or IV, wherein five of R¹, R², R³, R⁴, R⁵, or R⁶ are hydroxyl; and one of R¹, R², R³, R⁴, R⁵, or R⁶ is selected from CH₃, OCH₃, NO₂, CF₃, OCF₃, F, Cl, Br, I and CN.

In embodiments of the invention, the cyclohexanehexyl compound is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower alkyl, especially methyl, ethyl, butyl, or propyl, preferably methyl.

In embodiments of the invention, the cyclohexanehexyl compound is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower cycloalkyl, especially cyclopropyl, cyclobutyl, and cyclopentyl.

In embodiments of the invention, the cyclohexanehexyl compound is a compound of the formula I, II, III or IV wherein two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexyl compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexyl compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexyl compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, or R⁶ is halo, in particular fluoro, chloro or bromo, more particularly chloro.

In embodiments of the invention, the cyclohexanehexyl compound is a compound of the formula III or IV, wherein two, three, four or five of R¹, R², R³, R⁴, R⁵, or R⁶ are hydroxyl; at least one of R¹, R², R³, R⁴, R⁵, or R⁶ is halo; and the remainder of R¹, R², R³, R⁴, R⁵, or R⁶, if any, are independently C₁-C₆alkyl, C₂-C₆ alkenyl, C₂-C₆alkynyl, C₁C₆alkoxy, C₂-C₆alkenyloxy, C₃-C₁₀cycloalkyl, C₁-C₆acyl, C₁-C₆ acyloxy, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In still another aspect, the cyclohexanehexyl compound is a compound of formula III or IV, wherein four of R¹, R², R³, R⁴, R⁵, or R⁶ are hydroxyl; one of R¹, R², R³, R⁴, R⁵, or R⁶ is halo; and one of R¹, R², R³, R⁴, R⁵, or R⁶ is selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆ acyl, C₁-C₆ acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —Si(R⁷)₃, —CO₂R⁷, oxo, —PO₃H —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₆-C₁₀ heteroaryl and C₃-C₁₀heterocyclic, and at least one of R¹, R², R³, R⁴, R⁵, or R⁶ is halo.

In embodiments of the invention, the cyclohexanehexyl compound is a compound of the formula I, II, III or IV wherein five of R¹, R², R³, R⁴, R⁵, and/or R⁶ are hydroxyl and the other of R¹, R², R³, R⁴, R⁵, and/or R⁶ is halo, in particular fluoro, chloro or bromo, more particularly chloro.

In embodiments of the invention, the cyclohexanehexyl compound is a compound of the formula I, II, III or IV wherein R¹, R², R³, R⁴, and R⁵ are hydroxyl and R⁶ is halo, in particular fluorine, chlorine or bromine, more particularly chloro. In a particular embodiment of the invention, R¹, R², R³, R⁴, and R⁵ are hydroxyl and R⁶ is chloro.

In embodiments of the invention, the cyclohexanehexyl compound is a compound of the formula I, II, III or IV wherein R¹, R², R³, R⁴, and R⁶ are hydroxyl and R⁵ is halo, in particular fluoro, chloro or bromo, more particularly chloro. In a particular embodiment of the invention, R¹, R², R³, R⁴, and R⁶ are hydroxyl and R⁵ is chloro.

In embodiments of the invention, the cyclohexanehexyl compound is a compound of the formula I, II, III or IV wherein R¹, R², R³, R⁵, and R⁶ are hydroxyl and R⁴ is halo, in particular fluoro, chloro or bromo, more particularly chloro. In a particular embodiment of the invention, R¹, R², R³, R⁵, and R⁶ are hydroxyl and R⁴ is chloro.

In embodiments of the invention, the cyclohexanehexyl compound is a compound of the formula I, II, III or IV wherein R¹, R², R⁴, R⁵, and R⁶ are hydroxyl and R³ is halo, in particular fluoro, chloro or bromo, more particularly chloro. In a particular embodiment of the invention, R¹, R², R³, R⁴, R⁵, and R⁶ are hydroxyl and R³ is chloro.

In embodiments of the invention, the cyclohexanehexyl compound is a compound of the formula I, II, III or IV wherein R¹, R³, R⁴, R⁵, and R⁶ are hydroxyl and R² is halo, in particular fluoro, chloro or bromo, more particularly chloro. In a particular embodiment of the invention, R¹, R³, R⁴, R⁵, and R⁶ are hydroxyl and R² is chloro.

In embodiments of the invention, the cyclohexanehexyl compound is a compound of the formula I, II, III or IV wherein R², R³, R⁴, R⁵, and R⁶ are hydroxyl and R¹ is halo, in particular fluoro, chloro or bromo, more particularly chloro. In a particular embodiment of the invention, R², R³, R⁴, R⁵, and R⁶ are hydroxyl and R¹ is chloro.

In aspects of the invention, the cyclohexanehexyl compound is a scyllo-inositol compound, in particular a pure or substantially pure scyllo-inositol compound.

A “scyllo-inositol compound” includes compounds having the structure of the formula Va or Vb:

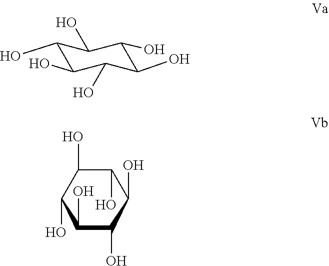

A scyllo-inositol compound includes a compound of the formula Va or Vb wherein one to six, one to five, one, two, three or four, preferably one, two or three, more preferably one or two hydroxyl groups are replaced by substituents, in particular univalent substituents, with retention of configuration. In aspects of the invention, a scyllo-inositol compound comprises a compound of the formula Va or Vb wherein one, two, three, four, five or six, preferably one or two, most preferably one, hydroxyl groups are replaced by univalent substituents, with retention of configuration. Suitable substituents include without limitation hydrogen; alkyl; substituted alkyl; acyl; alkenyl; substituted alkenyl; alkynyl; substituted alkynyl; cycloalkyl; substituted cycloalkyl; alkoxy; substituted alkoxy; aryl; aralkyl; substituted aryl; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; or —OR⁴⁵ wherein R⁴⁵ is hydrogen, alkyl, or —SO₃H.

In aspects of the invention, a scyllo-inositol compound does not include scyllo-cyclohexanehexyl substituted with one or more phosphate group.

Particular aspects of the invention utilize scyllo-inositol compounds of the formula Va or Vb wherein one or more of the hydroxyl groups is replaced with alkyl, in particular C₁-C₄ alkyl, more particularly methyl; acyl; chloro or fluoro; alkenyl; —NHR⁴¹ wherein R⁴¹ is hydrogen, acyl, alkyl or —R⁴²R⁴³ wherein R⁴² and R⁴³ are the same or different and represent acyl or alkyl; —SR⁴⁴ wherein R⁴⁴ is hydrogen, alkyl, or —O₃H; and —OR⁴⁵ wherein R⁴⁵ is hydrogen, alkyl, or —SO₃H, more particularly —SR⁴⁴ wherein R⁴⁴ is hydrogen, alkyl, or —O₃H or —OR⁴⁵ wherein R⁴⁵ is —SO₃H.

Particular aspects of the invention utilize scyllo-inositol compounds of the formula Va or Vb wherein one or more of the hydroxyl groups is replaced with alkyl; substituted alkyl; acyl; alkenyl; substituted alkenyl; —NHR⁴¹ wherein R⁴¹ is hydrogen, acyl, alkyl, or —R⁴²R⁴³ wherein R⁴² and R⁴³ are the same or different and represent acyl or alkyl; —SR⁴⁴ wherein R⁴⁴ is hydrogen, alkyl, or —O₃H; or —OR⁴⁵ wherein R⁴⁵ is hydrogen, alkyl or —SO₃H.

Particular aspects of the invention utilize scyllo-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; —OR⁴⁵ wherein R⁴⁵ is hydrogen, alkyl, or —OR⁴⁵ wherein R⁴⁵ is —SO₃H.

Particular aspects of the invention utilize scyllo-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; or thiol.

Particular aspects of the invention utilize scyllo-inositol compounds of the formula Va or Vb wherein one of the hydroxyl groups is replaced with alkyl, in particular C₁-C₄ alkyl, more particularly methyl.

Particular aspects of the invention utilize scyllo-inositol compounds of the formula Va or Vb wherein one of the hydroxyl groups is replaced with alkoxy, in particular C₁-C₄ alkoxy, more particularly methoxy or ethoxy, most particularly methoxy.

Particular aspects of the invention utilize scyllo-inositol compounds of the formula Va or Vb wherein one of the hydroxyl groups is replaced with halogen, in particular chloro or fluoro, more particularly fluoro.

Particular aspects of the invention utilize scyllo-inositol compounds of the formula Va or Vb wherein one of the hydroxyl groups is replaced with thiol.

In embodiments of the invention, the scyllo-inositol compound designated AZD-103/ELND005 (Elan Corporation) is used in the formulations, dosage forms, methods and uses disclosed herein.

In embodiments of the invention, the cyclohexanehexyl is O-methyl-scyllo-inositol

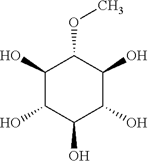

In embodiments of the invention, the cyclohexanehexyl is 1-chloro-1-deoxy-scyllo-inositol.

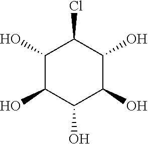

In aspects of the invention, the cyclohexanehexyl is an epi-inositol compound, in particular a pure or substantially pure epi-inositol compound.

An “epi-inositol compound” includes compounds having the base structure of formula VI:

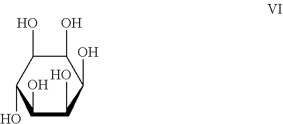

An epi-inositol compound includes a compound of the formula VI wherein one to six, one to five, one, two, three or four, preferably one, two or three, more preferably one or two hydroxyl groups are replaced by substituents, in particular univalent substituents, with retention of configuration. In aspects of the invention, an epi-inositol compound comprises a compound of the formula VI wherein one, two, three, four, five or six, preferably one or two, most preferably one, hydroxyl groups are replaced by univalent substituents, with retention of configuration. Suitable substituents include without limitation hydrogen; alkyl; substituted alkyl; acyl; alkenyl; substituted alkenyl; alkynyl; substituted alkynyl; cycloalkyl; substituted cycloalkyl; alkoxy; substituted alkoxy; aryl; aralkyl; substituted aryl; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; or —OR⁴⁵ wherein R⁴⁵ is hydrogen, alkyl, or —SO₃H.

Particular aspects of the invention utilize epi-inositol compounds of the formula VI wherein one or more of the hydroxyl groups is replaced with alkyl, in particular C₁-C₄ alkyl, more particularly methyl; acyl; chloro or fluoro; alkenyl; —NHR⁴¹ wherein R⁴¹ is hydrogen, acyl, alkyl or —R⁴²R⁴³ wherein R⁴² and R⁴³ are the same or different and represent acyl or alkyl; —SR⁴⁴ wherein R⁴⁴ is hydrogen, alkyl, or —O₃H; and —OR⁴⁵ wherein R⁴⁵ is hydrogen, alkyl, or —SO₃H, more particularly —SR⁴⁴ wherein R⁴⁴ is hydrogen, alkyl, or —O₃H or —OR⁴⁵ wherein R⁴⁵ is —SO₃H.

Particular aspects of the invention utilize epi-inositol compounds of the formula VI wherein one or more of the hydroxyl groups is replaced with alkyl; substituted alkyl; acyl; alkenyl; substituted alkenyl; —NHR⁴¹ wherein R⁴¹ is hydrogen, acyl, alkyl, or —R⁴²R⁴³ wherein R⁴² and R⁴³ are the same or different and represent acyl or alkyl; —SR⁴⁴ wherein R⁴⁴ is hydrogen, alkyl, or —O₃H; or —OR⁴⁵ wherein R⁴⁵ is hydrogen, alkyl or —SO₃H.

Particular aspects of the invention utilize epi-inositol compounds of the formula VI wherein one or more of the hydroxyl groups is replaced with alkyl; substituted alkyl; acyl; alkenyl; substituted alkenyl; alkynyl; substituted alkynyl; alkoxy; substituted alkoxy; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; —OR⁴⁵ wherein R⁴⁵ is hydrogen, alkyl, or —OR⁴⁵ wherein R⁴⁵ is —SO₃H.

Particular aspects of the invention utilize epi-inositol compounds of the formula VI wherein one or more of the hydroxyl groups is replaced with alkyl; substituted alkyl; acyl; alkenyl; substituted alkenyl; alkynyl; substituted alkynyl; alkoxy; substituted alkoxy; halogen; or thiol.

Particular aspects of the invention utilize epi-inositol compounds of the formula VI wherein one of the hydroxyl groups is replaced with alkyl, in particular C₁-C₄ alkyl, more particularly methyl.

Particular aspects of the invention utilize epi-inositol compounds of the formula VI wherein one of the hydroxyl groups is replaced with alkoxy, in particular C₁-C₄ alkoxy, more particularly methoxy or ethoxy, most particularly methoxy.

Particular aspects of the invention utilize epi-inositol compounds of the formula VI wherein one of the hydroxyl groups is replaced with halogen, in particular chloro or fluoro, more particularly fluoro.

Particular aspects of the invention utilize epi-inositol compounds of the formula VI wherein one of the hydroxyl groups is replaced with thiol.

In aspects of the invention, the cyclohexanehexyl is epi-inositol, in particular a pure or substantially pure epi-inositol.

Cyclohexanehexyl compounds utilized in the invention may be prepared using reactions and methods generally known to the person of ordinary skill in the art, having regard to that knowledge and the disclosure of this application. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing cyclohexanehexyl compounds are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and cyclohexanehexyl compounds may be isolated and purified using conventional techniques, such as precipitation, filtration, distillation, crystallization, chromatography, and the like. The compounds may be characterized using conventional methods, including physical constants and spectroscopic methods, in particular HPLC.

Cyclohexanehexyl compounds which are basic in nature can form a wide variety of different salts with various inorganic and organic acids. In practice it is desirable to first isolate a cyclohexanehexyl compound from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Cyclohexanehexyl compounds which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Scyllo-inositol compounds can be prepared using conventional processes or they may be obtained from commercial sources. For example, scyllo-inositol compounds can be prepared using chemical and/or microbial processes. In aspects of the invention, a scyllo-inositol is produced using process steps described by M. Sarmah and Shashidhar, M., Carbohydrate Research, 2003, 338, 999-1001, Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med. Chem. 1999, 7 (11):2577-89; or Kiely D. E., and Fletcher, H. G., J. American Chemical Society (US) 1968, 90:3289-3290; described in JP09-140388, DE 3,405,663 (Merck Patent GMBH), JP04-126075, JP05-192163, or WO06109479, or described in WO0503577, US20060240534, EP1674578, JP9140388, JP09140388, JP02-184912, JP03-102492 (Hokko Chemical Industries). In particular aspects of the compositions and methods of the invention, a scyllo-inositol is prepared using the chemical process steps described in Husson, C., et al, Carbohydrate Research 307 (1998) 163-165. In other aspects of the compositions and methods of the invention, a scyllo-inositol is prepared using microbial process steps similar to those described in WO05035774 (EP1674578 and US20060240534) JP2003102492, or JP09140388 (Hokko Chemical Industries). Derivatives may be produced by introducing substituents into a scyllo-inositol compound using methods well known to a person of ordinary skill in the art.

Epi-inositol compounds can be prepared using conventional processes or they may be obtained from commercial sources. In aspects of the invention, an epi-inositol compound can be prepared using chemical and/or microbial processes. For example, an epi-inositol compound may be prepared by the process described by V. Pistarà (Tetrahedron Letters 41, 3253, 2000), Magasanik B., and Chargaff E. (J Biol Chem, 1948, 174:173188), U.S. Pat. No. 7,157,268, or in PCT Published Application No. WO0075355. Derivatives may be produced by introducing substituents into an epi-inositol compound using methods well known to a person of ordinary skill in the art.

A cyclohexanehexyl compound may additionally comprise a carrier, including without limitation one or more of a polymer, carbohydrate, peptide or derivative thereof. A carrier may be substituted with substituents described herein including without limitation one or more alkyl, amino, nitro, halogen, thiol, thioalkyl, sulfate, sulfonyl, sulfenyl, sulfinyl, sulfoxide, hydroxyl groups. A carrier can be directly or indirectly covalently attached to a compound of the invention. In aspects of the invention the carrier is an amino acid including alanine, glycine, proline, methionine, serine, threonine, or asparagine. In other aspects the carrier is a peptide including alanyl-alanyl, prolyl-methionyl, or glycyl-glycyl.

A carrier also includes a molecule that targets a compound of the invention to a particular tissue or organ. In particular, a carrier may facilitate or enhance transport of a compound of the invention to the brain by either active or passive transport.

A “polymer” as used herein refers to molecules comprising two or more monomer subunits that may be identical repeating subunits or different repeating subunits. A monomer generally comprises a simple structure, low-molecular weight molecule containing carbon. Polymers can be optionally substituted. Examples of polymers which can be used in the present invention are vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In particular aspects of the invention, the polymer is poly(2-acrylamido-2-methyl-1-propanesulfonic acid), poly(2-acrylamido-2-methyl-1-propanesulfonic acid-coacrylonitrile, poly(2-acrylamido-2-methyl-1-propanesulfonic acid-co-styrene), poly(vinylsulfonic acid), poly(sodium 4-styrenesulfonic acid), and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The simplest carbohydrates are monosaccharides, which are small straight-chain aldehydes and ketones with many hydroxyl groups added, usually one on each carbon except the functional group. Examples of monosaccharides include erythrose, arabinose, allose, altrose, glucose, mannose, threose, xylose, gulose, idose, galactose, talose, aldohexose, fructose, ketohexose, ribose, and aldopentose. Other carbohydrates are composed of monosaccharide units, including disaccharides, oligosaccharides, or polysaccharides, depending on the number of monosaccharide units. Disaccharides are composed of two monosaccharide units joined by a covalent glycosidic bond. Examples of disaccharides are sucrose, lactose, and maltose. Oligosaccharides and polysaccharides, are composed of longer chains of monosaccharide units bound together by glycosidic bonds. Oligosaccharides generally contain between 3 and 9 monosaccharide units and polysaccharides contain greater than 10 monosaccharide units. A carbohydrate group may be substituted at one two, three or four positions, other than the position of linkage to a compound of the formula I, II, III or IV. For example, a carbohydrate may be substituted with one or more alkyl, amino, nitro, halo, thiol, carboxyl, or hydroxyl groups, which are optionally substituted. Illustrative substituted carbohydrates are glucosamine or galactosamine.

In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is selected from the group consisting of glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Suitable pentose sugars include arabinose, fucose, and ribose.

A sugar residue may be linked to a cyclohexanehexyl compound from a 1,1 linkage, 1,2 linkage, 1,3 linkage, 1,4 linkage, 1,5 linkage, or 1,6 linkage. A linkage may be via an oxygen atom of a cyclohexanehexyl compound. An oxygen atom can be replaced one or more times by —CH₂— or —S— groups.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” for use as a carrier in the practice of the present invention includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. In aspects of the invention the carrier is an amino acid such as alanine, glycine, proline, methionine, serine, threonine, histidine, or asparagine. In other aspects the carrier is a peptide such as alanyl-alanyl, prolyl-methionyl, or glycyl-glycyl. In still other aspects, the carrier is a polypeptide such as albumin, antitrypsin, macroglobulin, haptoglobin, caeruloplasm, transferrin, α- or β-lipoprotein, β- or γ-globulin or fibrinogen.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptide analogues, derivatives and peptidomimetics include peptides substituted with one or more benzodiazepine molecules (see e.g., James, G. L. et al. (1993) Science 260:1937-1942), peptides with methylated amide linkages and “retro-inverso” peptides (see U.S. Pat. No. 4,522,752 by Sisto).

Examples of peptide derivatives include peptides in which an amino acid side chain, the peptide backbone, or the amino- or carboxy-terminus has been derivatized (e.g., peptidic compounds with methylated amide linkages).

The term mimetic, and in particular, peptidomimetic, is intended to include isosteres. The term “isostere” refers to a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Other examples of isosteres include peptides substituted with one or more benzodiazepine molecules (see e.g., James, G. L. et al. (1993) Science 260:1937-1942)

Other possible modifications include an N-alkyl (or aryl) substitution ([CONR]), backbone crosslinking to construct lactams and other cyclic structures, substitution of all D-amino acids for all L-amino acids within the compound (“inverso” compounds) or retro-inverso amino acid incorporation ([NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A peptide can be attached to a compound of the invention through a functional group on the side chain of certain amino acids (e.g. serine) or other suitable functional groups. In embodiments of the invention the carrier may comprise four or more amino acids with groups attached to three or more of the amino acids through functional groups on side chains. In another embodiment, the carrier is one amino acid, in particular a sulfonate derivative of an amino acid, for example cysteic acid.

A “polyglutamine disease” refers to a condition or disorder associated with a disease protein (or fragment thereof) comprising polyglutamine (polyQ). The underlying mutation in a polyglutamine disease is an expansion CAG trinucleotide repeat that encodes polyglutamine in the disease protein. The diseases are characterized by mutant proteins which are unrelated except for the polyQ tract, and neuronal intranuclear and cytoplasmic containing aggregated polyQ. The number of glutamines observed in the pathological proteins may vary from 21 to >400 but typically a disease phenotype manifests above a repeat number varying between 35 and 40. The diseases are also characterized by late-onset, selective neuropathology, a pathogenic polyQ threshold and a relationship between polyQ length and disease progression. Examples of polyglutamine diseases include, without limitation, Huntington's disease (HD) and related neurodegenerative disorders, such as dentatorubral pallidoluysian atrophy (DRPLA), spinal and bulbar muscular atrophy (SBMA) and spinocerebellar ataxia (SCA) type 1, 2, 3, 6, 7, and 17.

### Medicaments

A cyclohexanehexyl compound or salts thereof as an active ingredient can be directly administered to a patient, but it is preferably administered as a preparation in the form of a medicament containing the active ingredient and pharmaceutically acceptable carriers, excipients, and vehicles. Therefore, the invention contemplates a medicament comprising a therapeutically effective amount of an isolated, in particular pure, cyclohexanehexyl compound, more particularly a scyllo-inositol compound or analog or derivative thereof, for treating a polyglutamine disease or symptoms caused by a polyglutamine disease and/or suppressing the progression of a polyglutamine disease.

Medicaments of the present invention or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21st Edition, Popovich, N (eds), Advanced Concepts Institute, University of the Sciences in Philadelphia, Philadelphia, Pa. 2005). A medicament of the invention can be in any form suitable for administration to a patient including, without limitation, a liquid solution, suspension, emulsion, tablet, pill, capsule, sustained release formulation, or powder.

Examples of preparations which are appropriate for oral administration can include capsules, tablets, powders, fine granules, solutions and syrups, where the active components can be combined with an oral, non-toxic pharmaceutically acceptable inert carrier such as lactose, starch, sucrose, cellulose, methyl cellulose, magnesium stearate, glucose, calcium sulfate, dicalcium phosphate, sodium saccharine, magnesium carbonate mannitol, sorbital, and the like. For oral administration in a liquid form, the active components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the medicaments or components thereof. Medicaments as described herein can further comprise wetting or emulsifying agents, or pH buffering agents.

Medicaments which are appropriate for parenteral administration may include aqueous solutions, syrups, aqueous or oil suspensions and emulsions with edible oil such as cottonseed oil, coconut oil or peanut oil. In aspects of the invention medicaments for parenteral administration include sterile aqueous or non-aqueous solvents, such as water, isotonic saline, isotonic glucose solution, buffer solution, or other solvents conveniently used for parenteral administration of therapeutically active agents. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone. A medicament intended for parenteral administration may also include conventional additives such as stabilizers, buffers, or preservatives, e.g. antioxidants such as methylhydroxybenzoate or similar additives.

Examples of additives for medicaments that can be used for injection or drip include a resolvent or a solubilizer that can compose an aqueous injection or an injection to be dissolved before use, such as distilled water for injection, physiological saline and propylene glycol, isotonizing agents such as glucose, sodium chloride, D-mannitol, and glycerine, and pH modifiers such as inorganic acid, organic acid, inorganic bases or organic base.

A medicament can be formulated as a suppository, with traditional binders and carriers such as triglycerides. Various known delivery systems can be used to administer a medicament of the invention, e.g. encapsulation in liposomes, microparticles, microcapsules, and the like. Medicaments can also be formulated as pharmaceutically acceptable salts as described herein.

A medicament can be sterilized by, for example, filtration through a bacteria retaining filter, addition of sterilizing agents to the medicament, irradiation of the medicament, or heating the medicament. Alternatively, the medicaments may be provided as sterile solid preparations e.g., lyophilized powder, which are readily dissolved in sterile solvent immediately prior to use.

A cyclohexanehexyl compound may be in a form suitable for administration as a dietary supplement. A supplement may optionally include inactive ingredients such as diluents or fillers, viscosity-modifying agents, preservatives, flavorings, colorants, or other additives conventional in the art. By way of example only, conventional ingredients such as beeswax, lecithin, gelatin, glycerin, caramel, and carmine may be included. A dietary supplement composition may optionally comprise a second active ingredient such as pinitol or an active derivative or metabolite thereof.

A dietary supplement may be provided as a liquid dietary supplement e.g., a dispensable liquid) or alternatively the compositions may be formulated as granules, capsules or suppositories. The liquid supplement may include a number of suitable carriers and additives including water, glycols, oils, alcohols, flavoring agents, preservatives, coloring agents and the like. In capsule, granule or suppository form, the dietary compositions are formulated in admixture with a pharmaceutically acceptable carrier.

A supplement may be presented in the form of a softgel which is prepared using conventional methods. A softgel typically includes a layer of gelatin encapsulating a small quantity of the supplement. A supplement may also be in the form of a liquid-filled and sealed gelatin capsule, which may be made using conventional methods.

To prepare a dietary supplement composition in capsule, granule or suppository form, one or more compositions comprising cyclohexanehexyl compounds may be intimately admixed with a pharmaceutically acceptable carrier according to conventional formulation techniques. For solid oral preparations such as capsules and granules, suitable carriers and additives such as starches, sugars, diluents, granulating agents, lubricants, binders, disintegrating agents and the like may be included.

According to the invention, a kit is provided. In an aspect, the kit comprises a cyclohexanehexyl compound or a medicament of the invention in kit form. The kit can be a package which houses a container which contains a cyclohexanehexyl compound or medicament of the invention and also houses instructions for administering the cyclohexanehexyl compound or medicament to a subject. The invention further relates to a commercial package comprising a cyclohexanehexyl compound or medicament together with instructions for simultaneous, separate or sequential use. In particular a label may include amount, frequency, and method of administration.

In embodiments of the invention, a pharmaceutical pack or kit is provided comprising one or more containers filled with one or more of the ingredients of a medicament of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The invention also relates to articles of manufacture and kits containing materials useful for treating a polyglutamine disease. An article of manufacture may comprise a container with a label. Examples of suitable containers include bottles, vials, and test tubes which may be formed from a variety of materials including glass and plastic. A container holds a medicament or formulation of the invention comprising a cyclohexanehexyl compound which is effective for treating a polyglutamine disease. The label on the container indicates that the medicament or formulation is used for treating a polyglutamine disease and may also indicate directions for use. In aspects of the invention, a medicament or formulation in a container may comprise any of the medicaments or formulations disclosed herein.

The invention also contemplates kits comprising one or more of a cyclohexanehexyl compound. In aspects of the invention, a kit of the invention comprises a container described herein. In particular aspects, a kit of the invention comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a polyglutamine disease). A medicament or formulation in a kit of the invention may comprise any of the formulations or compositions disclosed herein.

In aspects of the invention, the kits may be useful for any of the methods disclosed herein, including, without limitation treating a subject suffering from a polyglutamine disease. Kits of the invention may contain instructions for practicing any of the methods described herein.

### Methods

The invention contemplates the use of therapeutically effective amounts of a cyclohexanehexyl compound or medicament of the invention for treating a polyglutamine disease, in particular preventing, and/or ameliorating disease severity, disease symptoms, and/or periodicity of recurrence of a polyglutamine disease. The invention also contemplates treating in mammals a polyglutamine disease using the medicaments or treatments of the invention. Such uses and treatments may be effective for retarding the neurodegenerative effects of a polyglutamine disease.

According to the invention, a cyclohexanehexyl compound may be administered to any subject in the general population as prophylaxis against the possibility that the person may in the future develop a polyglutamine disease. In particular embodiments, a cyclohexanehexyl compound may be administered to a subject suspected of being at risk for a polyglutamine disease, for example, by virtue of being in a family with a higher than normal incidence of a polyglutamine disease or due to a defined genetic proclivity, for example as a result of a mutation in a disease gene such as the Huntingtin gene.

In an aspect, the invention provides use of a cyclohexanehexyl compound or medicament of the invention to prophylactically treat persons in the general population and more particularly persons believed to be at risk for developing a polyglutamine disease because of, for example, a positive family history for the disease and/or the presence of a genetic defect. In addition, a cyclohexanehexyl compound or a medicament of the invention may be used to treat persons already diagnosed with a polyglutamine disease to delay the progression of existing motor impairment and/or to delay the onset of motor impairment in motor systems not yet detectably affected by the disease.

In addition a cyclohexanehexyl compound may be administered to a subject in the early stages of a polyglutamine disease, in particular upon a determination that the diagnosis of a polyglutamine disease is probable. A period considered an “early stage” can be the first 6, 8, or 12 months after the onset of symptoms.

In aspects of the invention, a cyclohexanehexyl compound may be administered to a subject in the later stages to delay the onset of symptoms, in particular motor symptoms, for example, in order to delay impairment of vocalization and/or respiratory musculature associated with dysfunction of cranial motor nerves. A period considered a “later stage” can be more than 12 months after the onset of symptoms.

The medicaments and treatments of the invention preferably provide beneficial effects. In an embodiment, beneficial effects of a medicament or treatment of the invention can manifest as one or more or all of the following:
a) A reduction, slowing or prevention of an increase in, or an absence of symptoms of a polyglutamine disease, after administration to a subject with symptoms of a polyglutamine disease.
b) A reduction, slowing or prevention of an increase in, or an absence of neurodegenerative effects of a polyglutamine disease, including specifically, but not exclusively, degeneration of neuronal cells, especially in the frontal lobes, the basal ganglia, and the striatum.
c) A reduction, slowing or prevention of an increase in accumulation of PolyQ aggregates in neurons relative to the levels measured in the absence of a cyclohexanehexyl compound or medicament disclosed herein in subjects preferably with symptoms of a polyglutamine disease. In aspects of the invention, the cyclohexanehexyl compound or medicament induces at least about a 2%, 5%, 10%, 15%, 20%, 30%, 40%, 50%, 60%, 70%, 80%, or 90% decrease in accumulation of PolyQ aggregates.
d) A reduction in the kinetics of assembly of PolyQ aggregates, in particular a 2%, 5%, 10%, 15%, 20%, 30%, 40%, 50%, 60%, 70%, 80%, or 90% reduction in the kinetics of assembly of PolyQ aggregates.
e) A reduction, slowing or prevention of an increase in degeneration and death of neurons, in particular motor neurons, relative to the levels measured in the absence of a cyclohexanehexyl compound or medicament disclosed herein in subjects with symptoms of a polyglutamine disease. In aspects of the invention, the cyclohexanehexyl compound or medicament induces at least about a 2%, 5%, 10%, 15%, 20%, 30%, 40%, 50%, 60%, 70%, 80%, or 90% decrease in degeneration and death of neurons, in particular motor neurons, in the frontal lobes, the basal ganglia, and the striatum.
f) An increase or restoration of motor neuron function after administration to a subject with symptoms of a polyglutamine disease. In aspects of the invention a cyclohexanehexyl compound or medicament disclosed herein induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in motor neuron function in a subject.
g) A reduction or slowing of the rate of disease progression in a subject with a polyglutamine disease.
h) A reduction, slowing or prevention of motor neuron dysfunction. In aspects of the invention, the cyclohexanehexyl compound or medicament induces at least about a 2%, 5%, 10%, 15%, 20%, 30%, 40%, 50%, 60%, 70%, 80%, or 90% reduction or slowing or motor neuron dysfunction.
i) A reduction in accelerated mortality.
j) An increase in survival or longevity in a subject with symptoms of a polyglutamine disease.

In aspects of the invention beneficial effects of a medicament or treatment of the invention can manifest as (a) and (b); (a), (b) and (c); (a), (b), (c) and (d); (a), (b), (c), (d), (e) and (f); (a), (b), (c), (d), (e), (f) and (g); (a) to (h); (a) to (i); or (a) to (j).

Cyclohexanehexyl compounds, medicaments and methods of the invention can be selected that have sustained beneficial effects, preferably statistically significant sustained beneficial effects. In an embodiment, a medicament is provided comprising a therapeutically effective amount of a cyclohexanehexyl compound that provides a statistically significant sustained beneficial effect.

Greater efficacy and potency of a treatment of the invention in some aspects may improve the therapeutic ratio of treatment, reducing untoward side effects and toxicity. Selected methods of the invention may also improve long-standing polyglutamine disease even when treatment is begun long after the appearance of symptoms. Prolonged efficacious treatment can be achieved in accordance with the invention following administration of a cyclohexanehexyl compound or medicament comprising same.

In an aspect, the invention relates to a method for treating a polyglutamine disease comprising contacting PolyQ aggregates in a subject with a therapeutically effective amount of a cyclohexanehexyl compound or a medicament of the invention.

In another aspect, the invention provides a method for treating a polyglutamine disease by providing a medicament comprising a cyclohexanehexyl compound in an amount sufficient to disrupt PolyQ aggregates for a prolonged period following administration.

In a further aspect, the invention provides a method for treating a polyglutamine disease in a patient in need thereof which includes administering to the individual a medicament that provides a cyclohexanehexyl compound in a dose sufficient to increase motor neuron function. In another aspect, the invention provides a method for treating a polyglutamine disease comprising administering, preferably orally or systemically, an amount of a cyclohexanehexyl compound to a mammal, to reduce accumulation of PolyQ aggregates in neurons for a prolonged period following administration.

The invention in an embodiment provides a method for treating a polyglutamine disease, the method comprising administering to a mammal in need thereof a medicament comprising a cyclohexanehexyl compound in an amount sufficient to reduce motor neuron dysfunction for a prolonged period following administration, thereby treating the polyglutamine disease.

In another aspect, the invention provides a method for preventing and/or treating a polyglutamine disease, the method comprising administering to a mammal in need thereof a medicament comprising a cyclohexanehexyl compound in an amount sufficient to disrupt aggregated PolyQ for a prolonged period following administration; and determining the amount of aggregated PolyQ, thereby treating the polyglutamine disease. The amount of aggregated PolyQ may be measured using an antibody specific for PolyQ or a cyclohexanehexyl compound labeled with a detectable substance.

The present invention also includes methods of using the medicaments of the invention in combination with one or more additional therapeutic agents including without limitation agents that are used for the treatment of complications resulting from or associated with a polyglutamine disease, or general medications that treat or prevent side effects (e.g, anti-psychotics or reserpine).

The invention also contemplates the use of a medicament comprising at least one cyclohexanehexyl compound for treating a polyglutamine disease or in the preparation of a medicament for treating a polyglutamine disease. In an embodiment, the invention relates to the use of a therapeutically effective amount of at least one cyclohexanehexyl compound for treating a polyglutamine disease or in the preparation of a medicament for providing therapeutic effects, in particular beneficial effects, in treating a polyglutamine disease. In a still further embodiment the invention provides the use of a cyclohexanehexyl compound for prolonged or sustained treatment of a polyglutamine disease or in the preparation of a medicament for prolonged or sustained treatment of a polyglutamine disease.

Therapeutic efficacy and toxicity of medicaments and methods of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Medicaments which exhibit large therapeutic indices are preferred. By way of example, one or more of the therapeutic effects, in particular beneficial effects disclosed herein, can be demonstrated in a subject or disease model, for example, Huntington's disease models such as the cell culture and Drosophila models described in Zhang X et al, (2005, PNAS, 102:892-897), the HD transgenic mouse model (R6/2) described in Chou Sy et al, (J. Neurochem. 2005, 93 (2):310-20), and the transgenic mouse models of SBMA described in Katsuno M et al., (2003 Cytogenetic and Genome Research, 100:243-251).

### Administration

Cyclohexanehexyl compounds and medicaments for use in the present invention can be administered by any means that produce contact of the active agent(s) with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A cyclohexanehexyl compound and medicament for use in the invention can be formulated for sustained release, for delivery locally or systemically. It lies within the capability of a skilled physician or veterinarian to select a form and route of administration that optimizes the effects of the medicaments and treatments to provide therapeutic effects, in particular beneficial effects, more particularly sustained beneficial effects.

The cyclohexanehexyl compounds and medicaments may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The cyclohexanehexyl compounds and medicaments for use in the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

The dosage regimen of the invention will vary depending upon known factors such as the pharmacodynamic characteristics of the selected cyclohexanehexyl compounds and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a cyclohexanehexyl compound which will be effective in the treatment of a polyglutamine disease to provide effects, in particular beneficial effects, more particularly sustained beneficial effects, can be determined by standard clinical techniques. The precise dose to be employed in the formulation will also depend on the route of administration, and the seriousness of the disease, and will be decided according to the judgment of the practitioner and each patient's circumstances.

Suitable dosage ranges for administration are particularly selected to provide therapeutic effects, in particular beneficial effects, more particularly sustained beneficial effects. A dosage range is generally effective for triggering the desired biological responses. The dosage ranges may generally be about 0.01 μg to about 5 g per kg per day, about 0.1 μg to about 5 g per kg per day, about 0.1 mg to about 5 g per kg per day, about 0.1 mg to about 2 g per kg per day, about 0.5 mg to about 5 g per kg per day, about 1 mg to about 5 g per kg per day, about 1 mg to about 500 mg per kg per day, about 1 mg to about 200 mg per kg per day, about 1 mg to about 100 mg per kg per day, about 5 mg to about 100 mg per kg per day, about 10 mg to about 100 mg per kg, about 25 mg to about 75 mg per kg per day, about 1 mg to about 50 mg per kg per day, about 2 mg to about 50 mg/kg/day, about 2 mg to about 40 mg per kg per day, or about 3 mg to about 25 mg per kg per day. In aspects of the invention, the dosage ranges are generally about 0.01 μg to about 2 g per kg, about 1 μg to about 2 g per kg, about 1 mg to about 2 g per kg, 5 mg to about 2 g per kg, about 1 mg to about 1 g per kg, about 1 mg to about 200 mg per kg, about 1 mg to about 100 mg per kg, about 1 mg to about 50 mg per kg, about 10 mg to about 100 mg per kg, or about 25 mg to 75 mg per kg of the weight of a subject. A medicament or cyclohexanehexyl compound may be administered once, twice or more daily, in particular once daily.

In some aspects of the invention, the dosage ranges of a compound disclosed herein, administered once twice, three times or more daily, especially once or twice daily, are about 0.01 μg to 5 g/kg, 1 μg to 2 g/kg, 1 to 5 g/kg, 1 to 3 g/kg, 1 to 2 g/kg, 1 to 1 g/kg, 1 to 600 mg/kg, 1 to 500 mg/kg, 1 to 400 mg/kg, 1 to 200 mg/kg, 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 1 to 30 mg/kg, 3 to 30 mg/kg, 3 to 20 mg/kg, 1 to 20 mg/kg, or 1 to 15 mg/kg.

In embodiments of the invention, the required dose of a compound disclosed herein administered twice daily is about 1 to 50 mg/kg, 1 to 40 mg/kg, 2.5 to 40 mg/kg, 3 to 40 mg/kg, or 3 to 30 mg/kg. In embodiments of the invention, the required daily dose of the compound is about 0.01 μg to 5 g/kg, 1 μg to 5 mg/kg, or 1 mg to 1 g/kg and within that range 1 to 500 mg/kg, 1 to 250 mg/kg, 1 to 200 mg/kg, 1 to 150 mg/kg, 1 to 100 mg/kg, 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

In some aspects of the invention, the dosage ranges of a cyclohexanehexyl compound administered once twice, three times or more daily, especially once or twice daily, are about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In embodiments of the invention, the dosage ranges for the cyclohexanehexyl compound are about 0.1 mg to about 2 kg per kg per day, about 0.5 mg to about 2 g per kg per day, about 1 mg to about 1 g per kg per day, about 1 mg to about 200 mg per kg per day, about 1 mg to about 100 mg per kg per day, about 10 mg to about 100 mg per kg per day, about 30 mg to about 70 mg per kg per day, about 1 mg to about 50 mg per kg per day, about 2 mg to about 50 mg per kg per day, about 2 mg to about 40 mg per kg per day, or about 3 mg to 30 mg per kg per day.

In embodiments of the invention, the required dose of cyclohexanehexyl compound administered twice daily is about 1 to about 50 mg/kg, 1 to about 40 mg/kg, 2.5 to about 40 mg/kg, 3 to about 40 mg/kg, or 3 to about 35 mg/kg, in particular about 3 to about 30 mg/kg.

In other embodiments of the invention, the required daily dose of cyclohexanehexyl compound, is about 1 to about 80 mg/kg and within that range 1 to about 70 mg/kg, 1 to about 65 mg/kg, 2 to about 70 mg/kg, 3 to about 70 mg/kg, 4 to about 65 mg/kg, 5 to about 65 mg/kg, or 6 to about 60 mg/kg.

A cyclohexanehexyl compound can be provided once daily, twice daily, in a single dosage unit or multiple dosage units (i.e., tablets or capsules) having about 50 to about 10000 mg, 50 to about 2000 mg, 70 to about 7000 mg, 70 to about 6000 mg, 70 to about 5500 mg, 70 to about 5000 mg, 70 to about 4500 mg, 70 to about 4000 mg, 70 to about 3500 mg, 70 to about 3000 mg, 150 to about 2500 mg, 150 to about 2000 mg, 200 to about 2500, 200 to about 2000 mg, 200 to about 1500 mg, 700 to about 1200 mg, or 1000 mg, in particular 200 to 2000 mg, more particularly 700 to 1200 mg, most particularly 1000 mg.

In aspects of the invention, a cyclohexanehexyl compound is administered in an amount sufficient to result in peak plasma concentrations, Cₘₐₓ, of from or between about 1 to about 125 μg/ml, 1 to about 100 μg/ml, 1 to about 90 μg/ml, 1 to about 80 μg/ml, 1 to about 70 μg/ml, 1 to about 60 μg/ml, 1 to about 50 μg/ml, 1 to about 40 μg/ml, 1 to about 30 μg/ml, 1 to about 20 μg/ml, 1 to about 10 μg/ml, 1 to about 5 μg/ml, 5 to about 125 μg/ml, 5 to about 100 μg/ml, 5 to about 70 μg/ml, 5 to about 50 μg/ml, 10 to about 100 μg/ml, 10 to about 90 μg/ml, 10 to about 80 μg/ml, 10 to about 70 μg/ml, 10 to about 60 μg/ml, 10 to about 50 μg/ml, 10 to about 40 μg/ml, 10 to about 30 μg/ml, or 10 to about 20 μg/ml. In embodiments, the Cₘₐₓ, is between or from about 1-125 μg/ml, 1-100 μg/ml, 5-70 μg/ml, 5-50 μg/ml, 10-100 μg/ml, 10-90 μg/ml, 10-80 μg/ml, 10-70 μg/ml, 10-60 μg/ml, 10-50 μg/ml or 10-40 μg/ml. In particular embodiments, the Cₘₐₓ is from or between about 5 to about 70 μg/ml, 5 to about 65 μg/ml, 5 to about 50 μg/ml, 5 to about 40 μg/ml, 5 to about 30 μg/ml, or 5 to about 20 μg/ml.

The time to achieve a desirable plasma level (t_{1/2}) of a cyclohexanehexyl will depend on the individual treated, but is generally between about 1 to 200 hours, 1 to 150 hours, 1 to 125 hours, 1 to 100 hours, 1 to 80 hours, 1 to 70 hours, 1 to 50 hours, 1 to 42 hours, 1 to 33 hours, 3 to 50 hours, 16 to 32 hours, 5 to 30 hours, 10 to 30 hours, 1 to 28 hours, 1 to 25 hours, 10 to 25 hours, 1 to 24 hours, 10 to 24 hours, 13 to 24 hours, 1 to 23 hours, 1 to 20 hours, 1 to 18 hours, 1 to 15 hours, 1 to 14 hours, 1 to 13 hours, 1 to 12 hours, 1 to 10 hours, 1 to 8 hours, 1 to 7 hours, 1 to 5 hours, 1 to 4 hours, 1 to 3 hours or 3 to 5 hours, in particular 1 to 5 hours or 3 to 5 hours.

A medicament or treatment of the invention may comprise a unit dosage of at least one compound of the invention to provide beneficial effects. A “unit dosage” or “dosage unit” refers to a unitary, i.e. a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a cyclohexanehexyl compound or medicament thereof on substantially any desired schedule. A cyclohexanehexyl compound or medicament of the invention may be administered one or more times per day, in particular 1 or 2 times per day, once per week, once a month or continuously. However, a subject may be treated less frequently, such as every other day or once a week, or more frequently. A cyclohexanehexyl compound or medicament may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

In an aspect, the invention provides a regimen for supplementing a human's diet, comprising administering to the human a supplement comprising a cyclohexanehexyl compound or a nutraceutically acceptable derivative thereof. A subject may be treated with a supplement at least about every day, or less frequently, such as every other day or once a week. A supplement of the invention may be taken daily but consumption at lower frequency, such as several times per week or even isolated doses, may be beneficial. In a particular aspect, the invention provides a regimen for supplementing a human's diet, comprising administering to the human about 1 to about 1000, 5 to about 200 or about 25 to about 200 milligrams of a cyclohexanehexyl compound, or nutraceutically acceptable derivative thereof on a daily basis. In another aspect, about 50 to 100 milligrams of a cyclohexanehexyl compound is administered to the human on a daily basis.

A supplement of the present invention may be ingested with or after a meal. Thus, a supplement may be taken at the time of a person's morning meal, and/or at the time of a person's noontime meal. A portion may be administered shortly before, during, or shortly after the meal. For daily consumption, a portion of the supplement may be consumed shortly before, during, or shortly after the human's morning meal, and a second portion of the supplement may be consumed shortly before, during, or shortly after the human's noontime meal. The morning portion and the noontime portion can each provide approximately the same quantity of a cyclohexanehexyl compound. A supplement and regimens described herein may be most effective when combined with a balanced diet according to generally accepted nutritional guidelines, and a program of modest to moderate exercise several times a week.

In a particular aspect, a regimen for supplementing a human's diet is provided comprising administering to the human a supplement comprising, per gram of supplement: about 5 milligram to about 50 milligrams of one or more cyclohexanehexyl compound or a nutraceutically acceptable derivative thereof. In an embodiment, a portion of the supplement is administered at the time of the human's morning meal, and a second portion of the supplement is administered at the time of the human's noontime meal.

The invention will be described in greater detail by way of a specific example. The following example is offered for illustrative purposes, and is not intended to limit the invention in any manner.

### Example

### Introduction

Huntington's disease is a late onset hereditary neurodegenerative disease characterized by movement disorders, psychiatric symptoms and cognitive dysfunction (Beal, M. F. and Ferrante, R. J. (2004) Nature Rev Neuroscience 5, 373-384). The disease is characterized by an expansion of CAG repeat encoding an endogenous poly-glutamine repeat tract in the huntingtin protein. Both gain of function mutations associated with direct Htt protein toxicity and loss of function mutations of normal huntingtin have been proposed to contribute to Huntington's disease. The major pathological feature of this disease is the appearance of neuronal intranuclear inclusions containing mutant Htt protein as well as inclusions in neuronal processes. The length of the poly-Q repeat varies between families and the severity of the disease is linked to the length of the poly-Q tract, with the longer the tract the more severe the disease. Aggregation of mutant Htt is dependent on poly-Q tract length and protein concentration, while aging, proteosome dysfunction and chaperone activity deficits also contribute to aggregation. The events that are triggered directly by mutant huntingtin or its fragments trigger cascades of both damaging and compensatory molecular processes. These ultimately lead to increasing dysfunction of neurons that are then more susceptible to more generic stresses. Therefore, one mechanism to treat this disorder would be to target the initial aggregation of Htt and fragments prior to initiation of neuronal dysfunction, or to eliminate further insult once disease onset.

Mouse, rat, C. Elegans, drosophila, yeast and cell culture models have been developed to model different aspects of poly-Q repeat diseases (Beal, M. F. and Ferrante, R. J. (2004) Nature Rev Neuroscience 5, 373-384). In order to efficiently screen drug candidates and to study pathogenic mechanisms of disease, an inducible PC-12 cell model system was developed which recapitulates the aggregation of poly-Q proteins into inclusions and transcription dysregulation. A previous study demonstrated that disruption of aggregation in this model system correlated with in vivo rescue of neuronal degeneration in Drosophila model (Apotol, B. L. et al., (2003) PNAS 100, 5950-5955; Zhang, X. et al., (2005) PNAS 102, 892-897), thus suggesting that this was a viable approach to use for drug screening. Therefore this inducible cell line was used to determine the effects of scyllo-inositol on Htt aggregation, degradation and cell viability.

### Methods:

Inducible PC-12 cells. PC-12 cells stably transfected with pIND constructs containing HttQ103P-EGFP. The cells upon stimulation with 5 μM ponasterone for 48 hrs express poly-Q protein with a EGFP carboxy-terminal epitope tag (Apostol, B. et al., PNAS 100, 5950-5955, 2003).
Chemical Compound screen in PC-12 cells. Cells were plated in 6 well plates and grown overnight, then treated with 5 μM ponasterone for 48 hrs in the presence and absence of cystamine bitartrate or scyllo-inositol. Both were dissolved in water, and added directly to cultures at a final concentration of 1-50 μM and aggregation calculated. Visual counts of aggregates and EGFP-positive cells were performed using fluorescent microscopy. At least 300 cells were counted from 5-6 fields in three wells in each of three independent experiments. Aggregation is expressed as the percentage of cells with aggregates versus total number of EGFP-positive cells. Chemical compound was assessed for cellular toxicity by evaluating cell morphology, trypan blue exclusion and cell viability.
Protein Expression. Cells were plated in 6 well plates and grown overnight, then treated with 5 μM ponasterone for 48 hrs in the presence and absence of cystamine bitartrate or scyllo-inositol. Cells were lysed in hypotonic solution containing 20 mM HEPES ph 7.5, 5 mM NaCl, 10 mM NaF, 2 mM EDTA, 1% Nonidet P-40, 1 mM sodium, orthovanadate, and protease inhibitors. Protein concentration was determined using the Bradford assay and 100 μg of protein was used for western blotting. Primary antibody to poly-glutamine was used to detect HttQ103, while GAPDH was used as an internal house-keeping standard. Westerns were scanned and densitometry was used to quantify PolyQ expression.

### Results:

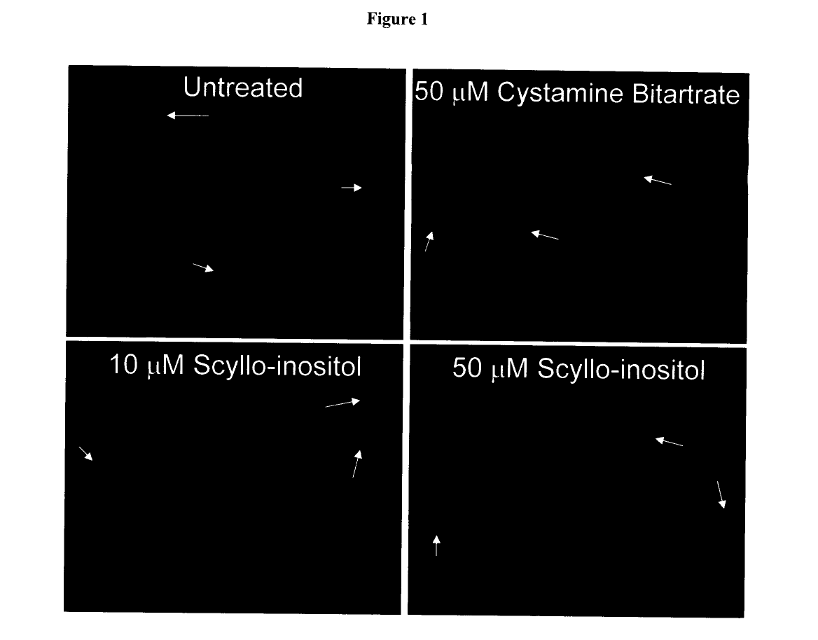

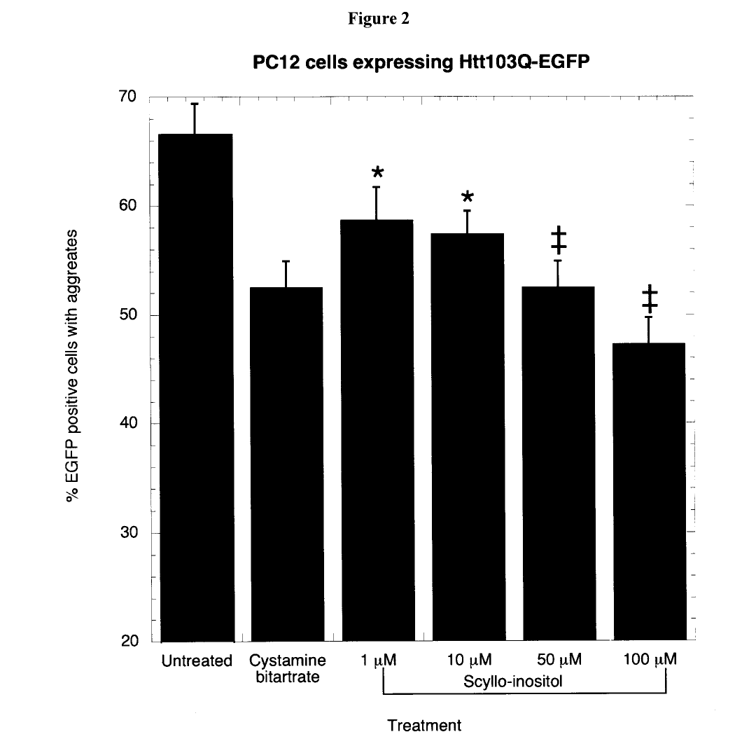

A hallmark of polyglutamine diseases is the accumulation of intracellular aggregates in the cytosol and nucleus of neurons. Therefore, the inducible PC-12 cell lines were utilized to address the question of inhibition of HttQ103P-EFGP aggregation using scyllo-inositol and a previously identified aggregation inhibitor, cystamine bitartrate. Aggregates are readily distinguished from soluble Htt, by intense small aggregate formation versus diffuse cellular staining pattern of soluble Htt (FIG. 1). Simultaneously with induction of HttQ103P-EFGP expression, scyllo-inositol was added to the cells in a final concentration range from 0-100 μM, with 50 μM cystamine bitartrate as a positive control. Cells were photographed using fluorescence microscopy and total cells expressing EFGP counted, and cells containing aggregates. Efficacy of treatment was determined by a reduction in the percentage of cells containing EGFP aggregates (FIG. 1, 2). Scyllo-inositol caused a concentration dependent decrease in the formation of aggregates, with significance detected as low as 1 μM scyllo-inositol concentration (FIG. 2). Morphological examination of cells did not detect changes associated with toxicity, further the trypan blue exclusion assay demonstrated that scyllo-inositol administration had no effect on cell viability.

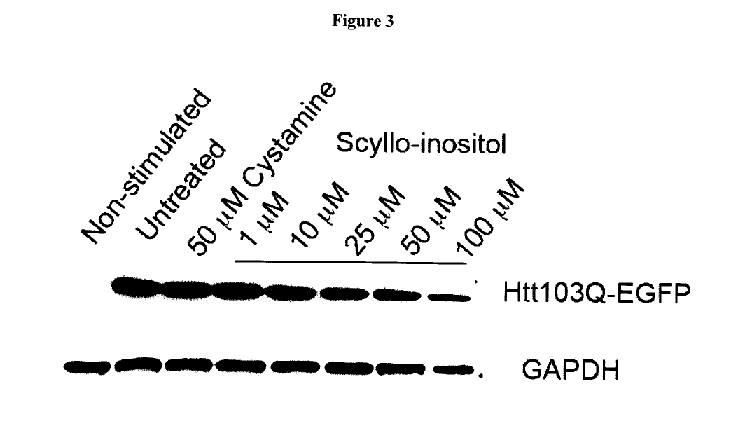

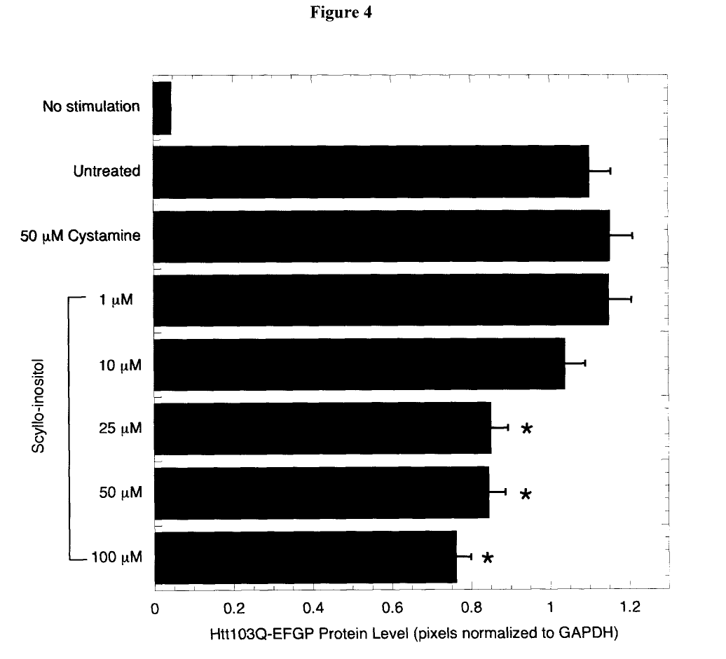

In order to examine changes in Htt103Q-EGFP protein concentration as a function of scyllo-inositol treatment, western blots were used (FIG. 3, 4). Cells were treated as above and then harvested for western blot analyses using a poly-glutamine specific antibody, and normalized to the housekeeping protein, GAPDH (FIG. 3). Blots were scanned and densitometry of each lane performed, to normalize for variations in protein loading pixels were normalized to those of GAPDH. Western blot demonstrated that although cystamine bitartrate inhibits aggregate formation, it has no effect on Htt103 cellular protein concentration (FIG. 4). In contrast, a concentration dependant decrease in Htt protein was detected. These results suggest that binding of scyllo-inositol to Htt not only inhibits aggregation but increases degradation of mutant Htt within the cell. This latter effect was seen at scyllo-inositol concentrations of 25-100 μM.

In conclusion, scyllo-inositol treatment decreases the aggregation of poly-Q repeat Htt at concentrations in the range of 1-100 μM, and at the higher concentrations within this range, 25-100 μM, also enhances mutant Htt degradation.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

What is claimed is:

1. A medicament comprising a therapeutically effective amount of a cyclohexanehexyl compound for treating a polyglutamine disease wherein the cyclohexanehexyl compound is a compound of formula III or IV 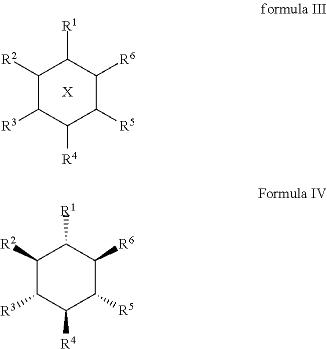
wherein X is a cyclohexane, R¹, R², R³, R⁴, R⁵, and R⁶ are hydroxyl or at least one of R¹, R², R³, R⁴, R⁵, and R⁶ is independently selected from hydrogen, C₁-C₆ alkyl, C₂-C₆ alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C₁₀ cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷, —NR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀ aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof.

2. A medicament according to claim 1 wherein the cyclohexanehexyl compound is a compound of the formula III or IV wherein four or five of R¹, R³, R⁴, R⁵, and R⁶ are hydroxyl; and one of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀ aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

3. A medicament according to claim 1 or 2 wherein the cyclohexanehexyl compound is a compound of the formula III or IV wherein one of R¹, R³, R⁴, R⁵, and R⁶ is C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

4. A medicament according to claim 1 wherein the cyclohexanehexyl compound is a compound of the formula III or IV wherein at least one, two, three or four of R¹, R³, R⁴, R⁵, and/or R⁶ are hydroxyl and the other of R¹, R³, R⁴, R⁵, and/or R⁶ are C₁-C₆ alkyl, C₁-C₆ alkoxy, or halo.

5. A medicament according to claim 1 wherein the cyclohexanehexyl compound is scyllo-inositol.

6. A medicament according to any one of claims 1 to 5 for use in the treatment of a polyglutamine disease characterized by PolyQ aggregate deposition.

7. A medicament according to any one of claims 1 to 6 wherein the amount of cyclohexanehexyl compound is effective to modulate assembly, folding, accumulation, rate of aggregation and/or clearance of proteins or fragments thereof comprising PolyQ.

8. A medicament according to any one of claims 1 to 6 wherein the amount of cyclohexanehexyl compound is effective to prevent, disrupt or inhibit assembly or reverse or reduce PolyQ aggregates after the onset of symptoms of a polyglutamine disease.

9. A medicament according to any one of claims 1 to 6 wherein the amount of cyclohexanehexyl compound is effective to improve or enhance motor neuron function and/or slow degeneration and death of motor neurons in the brain.

10. A method for modulating assembly, folding, accumulation, rate of aggregation and/or clearance of proteins or fragments thereof comprising PolyQ in a subject comprising administering a therapeutically effective amount of a medicament according to any one of claims 1 to 5.

11. A method for preventing or inhibiting assembly of, or reversing or reducing PolyQ aggregates in a subject after the onset of symptoms of a polyglutamine disease comprising administering a therapeutically effective amount of a medicament according to any one of claims 1 to 5.

12. A method for disrupting or enhancing clearance or degradation of PolyQ aggregates in a subject after the onset of symptoms of a polyglutamine disease comprising administering a therapeutically effective amount of a medicament according to any one of claims 1 to 5.

13. A method for improving motor neuron function and/or slowing degeneration or death of motor neurons in the brain in a subject after the onset of symptoms of a polyglutamine disease comprising administering a therapeutically effective amount of a medicament according to any one of claims 1 to 5.

14. A method for preventing, reducing and/or inhibiting in a subject PolyQ aggregate deposition comprising administering a therapeutically effective amount of a medicament according to any one of claims 1 to 5.

15. A method for ameliorating symptoms or onset of a polyglutamine disease in a subject comprising administering to the subject a therapeutically effective amount for ameliorating symptoms or onset of a polyglutamine disease of a medicament according to any one of claims 1 to 5.

16. A method of reversing PolyQ aggregate deposition and motor neuron death after the onset of symptoms in a subject suffering from a polyglutamine disease comprising administering to the subject a medicament according to any one of claims 1 to 5.

17. Use of a cyclohexanehexyl compound as defined in any one of claims 1 to 9 for treating a polyglutamine disease.

18. A kit comprising at least one medicament according to any one of claims 1 to 9, a container, and instructions for treating a polyglutamine disease.
